SEQUENCE LISTING (166 sequences)

SEQ ID NO: 1 - DNA, 19 bp, synthetic construct
  source 1..19: mol_type = other DNA
ttggataggcaggtggact

SEQ ID NO: 2 - DNA, 19 bp, synthetic construct
  source 1..19: mol_type = other DNA
tgcactgagaatactgtcc

SEQ ID NO: 3 - DNA, 19 bp, synthetic construct
  source 1..19: mol_type = other DNA
tcaacaaggagtacccggg

SEQ ID NO: 4 - DNA, 19 bp, synthetic construct
  source 1..19: mol_type = other DNA
taggagagcactgagaata

SEQ ID NO: 5 - DNA, 19 bp, synthetic construct
  source 1..19: mol_type = other DNA
tcttgtccagctttattgg

SEQ ID NO: 6 - DNA, 19 bp, synthetic construct
  source 1..19: mol_type = other DNA
ttccagctttattgggagg

SEQ ID NO: 7 - DNA, 19 bp, synthetic construct
  source 1..19: mol_type = other DNA
tataggcaggtggacttgg

SEQ ID NO: 8 - DNA, 19 bp, synthetic construct
  source 1..19: mol_type = other DNA
tgtccagctttattgggag

SEQ ID NO: 9 - DNA, 19 bp, synthetic construct
  source 1..19: mol_type = other DNA
tcactgagaatactgtccc

SEQ ID NO: 10 - DNA, 19 bp, synthetic construct
  source 1..19: mol_type = other DNA
tgagaatactgtccctttt

SEQ ID NO: 11 - DNA, 19 bp, synthetic construct
  source 1..19: mol_type = other DNA
tcaaggagctcgcaggatg

SEQ ID NO: 12 - DNA, 19 bp, synthetic construct
  source 1..19: mol_type = other DNA
tgcttcttgtccagcttta

SEQ ID NO: 13 - DNA, 19 bp, synthetic construct
  source 1..19: mol_type = other DNA
tggatggataggcaggtgg

SEQ ID NO: 14 - DNA, 19 bp, synthetic construct
  source 1..19: mol_type = other DNA
tcagaactcagagaacttg

SEQ ID NO: 15 - DNA, 19 bp, synthetic construct
  source 1..19: mol_type = other DNA
tctgagaatactgtccctt

SEQ ID NO: 16 - DNA, 19 bp, synthetic construct
  source 1..19: mol_type = other DNA
tctcgcaggatggataggc

SEQ ID NO: 17 - DNA, 19 bp, synthetic construct
  source 1..19: mol_type = other DNA
tcctgcacgctgctcagtg

SEQ ID NO: 18 - DNA, 19 bp, synthetic construct
  source 1..19: mol_type = other DNA
tgctccagtagtctttcag

SEQ ID NO: 19 - DNA, 19 bp, synthetic construct
  source 1..19: mol_type = other DNA
tatactgtcccttttaagc

SEQ ID NO: 20 - DNA, 19 bp, synthetic construct
  source 1..19: mol_type = other DNA
tggaactgaagccatcggt

SEQ ID NO: 21 - DNA, 19 bp, synthetic construct
  source 1..19: mol_type = other DNA
ttctcaggcagccacggct

SEQ ID NO: 22 - DNA, 19 bp, synthetic construct
  source 1..19: mol_type = other DNA
tcacggctgaagttggtct

SEQ ID NO: 23 - DNA, 19 bp, synthetic construct
  source 1..19: mol_type = other DNA
tagtctttcagggaactga

SEQ ID NO: 24 - DNA, 19 bp, synthetic construct
  source 1..19: mol_type = other DNA
tatactgtcccttttaagc

SEQ ID NO: 25 - DNA, 19 bp, synthetic construct
  source 1..19: mol_type = other DNA
tggtattgaggtctcaggc

SEQ ID NO: 26 - DNA, 19 bp, synthetic construct
  source 1..19: mol_type = other DNA
ttcagtgcatccttggcgg

SEQ ID NO: 27 - DNA, 19 bp, synthetic construct
  source 1..19: mol_type = other DNA
tatcccagaactcagagaa

SEQ ID NO: 28 - DNA, 19 bp, synthetic construct
  source 1..19: mol_type = other DNA
tagccatcggtcacccagc

SEQ ID NO: 29 - DNA, 19 bp, synthetic construct
  source 1..19: mol_type = other DNA
tgacccaaggagctcgcag

SEQ ID NO: 30 - DNA, 19 bp, synthetic construct
  source 1..19: mol_type = other DNA
ttccagtagtctttcaggg

SEQ ID NO: 31 - DNA, 15 bp, synthetic construct
  source 1..15: mol_type = other DNA
cacctgcctatccaa

SEQ ID NO: 32 - DNA, 15 bp, synthetic construct
  source 1..15: mol_type = other DNA
agtattctcagtgca

SEQ ID NO: 33 - DNA, 15 bp, synthetic construct
  source 1..15: mol_type = other DNA
ggtactccttgttga

SEQ ID NO: 34 - DNA, 15 bp, synthetic construct
  source 1..15: mol_type = other DNA
ctcagtgctctccta

SEQ ID NO: 35 - DNA, 15 bp, synthetic construct
  source 1..15: mol_type = other DNA
taaagctggacaaga

SEQ ID NO: 36 - DNA, 15 bp, synthetic construct
  source 1..15: mol_type = other DNA
ccaataaagctggaa

SEQ ID NO: 37 - DNA, 15 bp, synthetic construct
  source 1..15: mol_type = other DNA
gtccacctgcctata

SEQ ID NO: 38 - DNA, 15 bp, synthetic construct
  source 1..15: mol_type = other DNA
caataaagctggaca

SEQ ID NO: 39 - DNA, 15 bp, synthetic construct
  source 1..15: mol_type = other DNA
cagtattctcagtga

SEQ ID NO: 40 - DNA, 15 bp, synthetic construct
  source 1..15: mol_type = other DNA
gggacagtattctca

SEQ ID NO: 41 - DNA, 15 bp, synthetic construct
  source 1..15: mol_type = other DNA
ctgcgagctccttga

SEQ ID NO: 42 - DNA, 15 bp, synthetic construct
  source 1..15: mol_type = other DNA
gctggacaagaagca

SEQ ID NO: 43 - DNA, 15 bp, synthetic construct
  source 1..15: mol_type = other DNA
ctgcctatccatcca

SEQ ID NO: 44 - DNA, 15 bp, synthetic construct
  source 1..15: mol_type = other DNA
ttctctgagttctga

SEQ ID NO: 45 - DNA, 15 bp, synthetic construct
  source 1..15: mol_type = other DNA
gacagtattctcaga

SEQ ID NO: 46 - DNA, 15 bp, synthetic construct
  source 1..15: mol_type = other DNA
atccatcctgcgaga

SEQ ID NO: 47 - DNA, 15 bp, synthetic construct
  source 1..15: mol_type = other DNA
gagcagcgtgcagga

SEQ ID NO: 48 - DNA, 15 bp, synthetic construct
  source 1..15: mol_type = other DNA
aagactactggagca

SEQ ID NO: 49 - DNA, 15 bp, synthetic construct
  source 1..15: mol_type = other DNA
aaaagggacagtata

SEQ ID NO: 50 - DNA, 15 bp, synthetic construct
  source 1..15: mol_type = other DNA
atggcttcagttcca

SEQ ID NO: 51 - DNA, 15 bp, synthetic construct
  source 1..15: mol_type = other DNA
gtggctgcctgagaa

SEQ ID NO: 52 - DNA, 15 bp, synthetic construct
  source 1..15: mol_type = other DNA
caacttcagccgtga

SEQ ID NO: 53 - DNA, 15 bp, synthetic construct
  source 1..15: mol_type = other DNA
ttccctgaaagacta

SEQ ID NO: 54 - DNA, 15 bp, synthetic construct
  source 1..15: mol_type = other DNA
aaaagggacagtata

SEQ ID NO: 55 - DNA, 15 bp, synthetic construct
  source 1..15: mol_type = other DNA
gagacctcaatacca

SEQ ID NO: 56 - DNA, 15 bp, synthetic construct
  source 1..15: mol_type = other DNA
caaggatgcactgaa

SEQ ID NO: 57 - DNA, 15 bp, synthetic construct
  source 1..15: mol_type = other DNA
ctgagttctgggata

SEQ ID NO: 58 - DNA, 15 bp, synthetic construct
  source 1..15: mol_type = other DNA
ggtgaccgatggcta

SEQ ID NO: 59 - DNA, 15 bp, synthetic construct
  source 1..15: mol_type = other DNA
gagctccttgggtca

SEQ ID NO: 60 - DNA, 15 bp, synthetic construct
  source 1..15: mol_type = other DNA
gaaagactactggaa

SEQ ID NO: 61 - DNA, 19 bp, synthetic construct
  source 1..19: mol_type = other DNA
ttagaagaaaaggtgggag

SEQ ID NO: 62 - DNA, 19 bp, synthetic construct
  source 1..19: mol_type = other DNA
gttgagggagttttgctgg

SEQ ID NO: 63 - DNA, 19 bp, synthetic construct
  source 1..19: mol_type = other DNA
aaacactggttcttgcctc

SEQ ID NO: 64 - DNA, 19 bp, synthetic construct
  source 1..19: mol_type = other DNA
ccgcgctaaacactggttc

SEQ ID NO: 65 - DNA, 19 bp, synthetic construct
  source 1..19: mol_type = other DNA
ctggaaagtgagaccctcc

SEQ ID NO: 66 - DNA, 19 bp, synthetic construct
  source 1..19: mol_type = other DNA
ttcacaaacaagctggtcg

SEQ ID NO: 67 - DNA, 19 bp, synthetic construct
  source 1..19: mol_type = other DNA
ttttgttctcaacttgaaa

SEQ ID NO: 68 - DNA, 19 bp, synthetic construct
  source 1..19: mol_type = other DNA
ggtactctcattgtggatg

SEQ ID NO: 69 - DNA, 19 bp, synthetic construct
  source 1..19: mol_type = other DNA
actctgtgggctctctctc

SEQ ID NO: 70 - DNA, 19 bp, synthetic construct
  source 1..19: mol_type = other DNA
acaggtactctcattgtgg

SEQ ID NO: 71 - DNA, 19 bp, synthetic construct
  source 1..19: mol_type = other DNA
caaacaagctggtcggttg

SEQ ID NO: 72 - DNA, 19 bp, synthetic construct
  source 1..19: mol_type = other DNA
gggctctctctcatccgct

SEQ ID NO: 73 - DNA, 19 bp, synthetic construct
  source 1..19: mol_type = other DNA
cagcaaacaggaatgggcg

SEQ ID NO: 74 - DNA, 19 bp, synthetic construct
  source 1..19: mol_type = other DNA
cttgaaaagggaacacttt

SEQ ID NO: 75 - DNA, 19 bp, synthetic construct
  source 1..19: mol_type = other DNA
ctttaattttaaaacccaa

SEQ ID NO: 76 - DNA, 19 bp, synthetic construct
  source 1..19: mol_type = other DNA
atgaacctgtcaatcttct

SEQ ID NO: 77 - DNA, 19 bp, synthetic construct
  source 1..19: mol_type = other DNA
acaaacaagctggtcggtt

SEQ ID NO: 78 - DNA, 19 bp, synthetic construct
  source 1..19: mol_type = other DNA
caagctggtcggttggaat

SEQ ID NO: 79 - DNA, 19 bp, synthetic construct
  source 1..19: mol_type = other DNA
gtagactctgtgggctctc

SEQ ID NO: 80 - DNA, 19 bp, synthetic construct
  source 1..19: mol_type = other DNA
tctcaacttgaaaagggaa

SEQ ID NO: 81 - DNA, 19 bp, synthetic construct
  source 1..19: mol_type = other DNA
aaaatgctgttcagcacct

SEQ ID NO: 82 - DNA, 19 bp, synthetic construct
  source 1..19: mol_type = other DNA
gagggagttttgctggaaa

SEQ ID NO: 83 - DNA, 19 bp, synthetic construct
  source 1..19: mol_type = other DNA
agagaggccagggtgccaa

SEQ ID NO: 84 - DNA, 19 bp, synthetic construct
  source 1..19: mol_type = other DNA
ccttctgctgtagtaccca

SEQ ID NO: 85 - DNA, 19 bp, synthetic construct
  source 1..19: mol_type = other DNA
cggaagcccaagaagttgg

SEQ ID NO: 86 - DNA, 19 bp, synthetic construct
  source 1..19: mol_type = other DNA
ttccatcctgtcacagcct

SEQ ID NO: 87 - DNA, 19 bp, synthetic construct
  source 1..19: mol_type = other DNA
aaacaagctggtcggttgg

SEQ ID NO: 88 - DNA, 19 bp, synthetic construct
  source 1..19: mol_type = other DNA
tgaggtgctgttgtccacc

SEQ ID NO: 89 - DNA, 19 bp, synthetic construct
  source 1..19: mol_type = other DNA
cactgaggtgctgttgtcc

SEQ ID NO: 90 - DNA, 19 bp, synthetic construct
  source 1..19: mol_type = other DNA
ggtgctgttgtccacccag

SEQ ID NO: 91 - DNA, 14 bp, synthetic construct
  source 1..14: mol_type = other DNA
accttttcttctaa

SEQ ID NO: 92 - DNA, 14 bp, synthetic construct
  source 1..14: mol_type = other DNA
aaaactccctcaac

SEQ ID NO: 93 - DNA, 14 bp, synthetic construct
  source 1..14: mol_type = other DNA
aagaaccagtgttt

SEQ ID NO: 94 - DNA, 14 bp, synthetic construct
  source 1..14: mol_type = other DNA
agtgtttagcgcgg

SEQ ID NO: 95 - DNA, 14 bp, synthetic construct
  source 1..14: mol_type = other DNA
gtctcactttccag

SEQ ID NO: 96 - DNA, 14 bp, synthetic construct
  source 1..14: mol_type = other DNA
agcttgtttgtgaa

SEQ ID NO: 97 - DNA, 14 bp, synthetic construct
  source 1..14: mol_type = other DNA
agttgagaacaaaa

SEQ ID NO: 98 - DNA, 14 bp, synthetic construct
  source 1..14: mol_type = other DNA
acaatgagagtacc

SEQ ID NO: 99 - DNA, 14 bp, synthetic construct
  source 1..14: mol_type = other DNA
agagcccacagagt

SEQ ID NO: 100 - DNA, 14 bp, synthetic construct
  source 1..14: mol_type = other DNA
atgagagtacctgt

SEQ ID NO: 101 - DNA, 14 bp, synthetic construct
  source 1..14: mol_type = other DNA
gaccagcttgtttg

SEQ ID NO: 102 - DNA, 14 bp, synthetic construct
  source 1..14: mol_type = other DNA
atgagagagagccc

SEQ ID NO: 103 - DNA, 14 bp, synthetic construct
  source 1..14: mol_type = other DNA
attcctgtttgctg

SEQ ID NO: 104 - DNA, 14 bp, synthetic construct
  source 1..14: mol_type = other DNA
gttcccttttcaag

SEQ ID NO: 105 - DNA, 14 bp, synthetic construct
  source 1..14: mol_type = other DNA
ttttaaaattaaag

SEQ ID NO: 106 - DNA, 14 bp, synthetic construct
  source 1..14: mol_type = other DNA
attgacaggttcat

SEQ ID NO: 107 - DNA, 14 bp, synthetic construct
  source 1..14: mol_type = other DNA
accagcttgtttgt

SEQ ID NO: 108 - DNA, 14 bp, synthetic construct
  source 1..14: mol_type = other DNA
aaccgaccagcttg

SEQ ID NO: 109 - DNA, 14 bp, synthetic construct
  source 1..14: mol_type = other DNA
cccacagagtctac

SEQ ID NO: 110 - DNA, 14 bp, synthetic construct
  source 1..14: mol_type = other DNA
ttttcaagttgaga

SEQ ID NO: 111 - DNA, 14 bp, synthetic construct
  source 1..14: mol_type = other DNA
ctgaacagcatttt

SEQ ID NO: 112 - DNA, 14 bp, synthetic construct
  source 1..14: mol_type = other DNA
agcaaaactccctc

SEQ ID NO: 113 - DNA, 14 bp, synthetic construct
  source 1..14: mol_type = other DNA
accctggcctctct

SEQ ID NO: 114 - DNA, 14 bp, synthetic construct
  source 1..14: mol_type = other DNA
actacagcagaagg

SEQ ID NO: 115 - DNA, 14 bp, synthetic construct
  source 1..14: mol_type = other DNA
ttcttgggcttccg

SEQ ID NO: 116 - DNA, 14 bp, synthetic construct
  source 1..14: mol_type = other DNA
gtgacaggatggaa

SEQ ID NO: 117 - DNA, 14 bp, synthetic construct
  source 1..14: mol_type = other DNA
cgaccagcttgttt

SEQ ID NO: 118 - DNA, 14 bp, synthetic construct
  source 1..14: mol_type = other DNA
acaacagcacctca

SEQ ID NO: 119 - DNA, 14 bp, synthetic construct
  source 1..14: mol_type = other DNA
acagcacctcagtg

SEQ ID NO: 120 - DNA, 14 bp, synthetic construct
  source 1..14: mol_type = other DNA
tggacaacagcacc

SEQ ID NO: 121 - DNA, 33 bp, synthetic construct
  source 1..33: mol_type = other DNA
ttagaagaaaaggtgggagaccttttcttctaa

SEQ ID NO: 122 - DNA, 33 bp, synthetic construct
  source 1..33: mol_type = other DNA
ttcacaaacaagctggtcgagcttgtttgtgaa

SEQ ID NO: 123 - DNA, 33 bp, synthetic construct
  source 1..33: mol_type = other DNA
ttttgttctcaacttgaaaagttgagaacaaaa

SEQ ID NO: 124 - DNA, 33 bp, synthetic construct
  source 1..33: mol_type = other DNA
tagcaaacaggaatgggcgattcctgtttgcta

SEQ ID NO: 125 - DNA, 33 bp, synthetic construct
  source 1..33: mol_type = other DNA
tgtactctcattgtggatgacaatgagagtaca

SEQ ID NO: 126 - DNA, 33 bp, synthetic construct
  source 1..33: mol_type = other DNA
ttgaacctgtcaatcttctattgacaggttcaa

SEQ ID NO: 127 - DNA, 33 bp, synthetic construct
  source 1..33: mol_type = other DNA
atagaagaaaaggtgggagaccttttcttctat

SEQ ID NO: 128 - DNA, 33 bp, synthetic construct
  source 1..33: mol_type = other DNA
atcacaaacaagctggtcgagcttgtttgtgat

SEQ ID NO: 129 - DNA, 33 bp, synthetic construct
  source 1..33: mol_type = other DNA
atttgttctcaacttgaaaagttgagaacaaat

SEQ ID NO: 130 - DNA, 34 bp, synthetic construct
  source 1..34: mol_type = other DNA
tcaacaaggagtacccgggcaccttttcttctaa

SEQ ID NO: 131 - DNA, 34 bp, synthetic construct
  source 1..34: mol_type = other DNA
ttagaagaaaaggtgggagggtactccttgttga

SEQ ID NO: 132 - DNA, 34 bp, synthetic construct
  source 1..34: mol_type = other DNA
tcaacaaggagtacccgggcagcttgtttgtgaa

SEQ ID NO: 133 - DNA, 34 bp, synthetic construct
  source 1..34: mol_type = other DNA
ttcacaaacaagctggtcgggtactccttgttga

SEQ ID NO: 134 - DNA, 34 bp, synthetic construct
  source 1..34: mol_type = other DNA
tcaacaaggagtacccgggaagttgagaacaaaa

SEQ ID NO: 135 - DNA, 34 bp, synthetic construct
  source 1..34: mol_type = other DNA
ttttgttctcaacttgaaaggtactccttgttga

SEQ ID NO: 136 - DNA, 34 bp, synthetic construct
  source 1..34: mol_type = other DNA
tcaacaaggagtacccggggattgacaggttcaa

SEQ ID NO: 137 - DNA, 34 bp, synthetic construct
  source 1..34: mol_type = other DNA
ttgaacctgtcaatcttctggtactccttgttga

SEQ ID NO: 138 - DNA, 34 bp, synthetic construct
  source 1..34: mol_type = other DNA
tcaacaaggagtacccgggcattcctgtttgcta

SEQ ID NO: 139 - DNA, 34 bp, synthetic construct
  source 1..34: mol_type = other DNA
tagcaaacaggaatgggcgggtactccttgttga

SEQ ID NO: 140 - DNA, 34 bp, synthetic construct
  source 1..34: mol_type = other DNA
tcaacaaggagtacccgggcacaatgagagtaca

SEQ ID NO: 141 - DNA, 34 bp, synthetic construct
  source 1..34: mol_type = other DNA
tgtactctcattgtggatgggtactccttgttga

SEQ ID NO: 142 - DNA, 34 bp, synthetic construct
  source 1..34: mol_type = other DNA
tcaacaaggagtacccgggcaccttttcttctat

SEQ ID NO: 143 - DNA, 34 bp, synthetic construct
  source 1..34: mol_type = other DNA
atagaagaaaaggtgggagggtactccttgttga

SEQ ID NO: 144 - DNA, 34 bp, synthetic construct
  source 1..34: mol_type = other DNA
tcaacaaggagtacccgggcagcttgtttgtgat

SEQ ID NO: 145 - DNA, 34 bp, synthetic construct
  source 1..34: mol_type = other DNA
atcacaaacaagctggtcgggtactccttgttga

SEQ ID NO: 146 - DNA, 34 bp, synthetic construct
  source 1..34: mol_type = other DNA
tcaacaaggagtacccgggaagttgagaacaaat

SEQ ID NO: 147 - DNA, 34 bp, synthetic construct
  source 1..34: mol_type = other DNA
atttgttctcaacttgaaaggtactccttgttga

SEQ ID NO: 148 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
ttagaagaaaaggtgggagggtactccttgttga

SEQ ID NO: 149 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
tcaacaaggagtacccgggcaccttttcttctaa

SEQ ID NO: 150 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
ttcacaaacaagctggtcgggtactccttgttga

SEQ ID NO: 151 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
tcaacaaggagtacccgggcagcttgtttgtgaa

SEQ ID NO: 152 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
ttttgttctcaacttgaaaggtactccttgttga

SEQ ID NO: 153 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
tcaacaaggagtacccgggaagttgagaacaaaa

SEQ ID NO: 154 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
ttgaacctgtcaatcttctggtactccttgttga

SEQ ID NO: 155 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
tcaacaaggagtacccggggattgacaggttcaa

SEQ ID NO: 156 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
tagcaaacaggaatgggcgggtactccttgttga

SEQ ID NO: 157 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
tcaacaaggagtacccgggcattcctgtttgcta

SEQ ID NO: 158 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
tgtactctcattgtggatgggtactccttgttga

SEQ ID NO: 159 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
tcaacaaggagtacccgggcacaatgagagtaca

SEQ ID NO: 160 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
atagaagaaaaggtgggagggtactccttgttga

SEQ ID NO: 161 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
tcaacaaggagtacccgggcaccttttcttctat

SEQ ID NO: 162 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
atcacaaacaagctggtcgggtactccttgttga

SEQ ID NO: 163 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
tcaacaaggagtacccgggcagcttgtttgtgat

SEQ ID NO: 164 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
atttgttctcaacttgaaaggtactccttgttga

SEQ ID NO: 165 - RNA, 34 nt, synthetic construct
  source 1..34: mol_type = other RNA
  modified_base 1: mod_base = OTHER; note = 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 20: mod_base = OTHER; note = 2'-fluoro modified nucleotide with phosphorothioate linkage
  modified_base 21: mod_base = OTHER; note = 2'-O-methyl modified nucleotide with phosphorothioate linkage
  modified_base 22: mod_base = OTHER; note = 2'-fluoro modified nucleotide
  modified_base 23: mod_base = OTHER; note = 2'-O-methyl modified nucleotide
  modified_base 24: mod_base = OTHER; note = 2'-fluoro modified nucleotide
  modified_base 25: mod_base = OTHER; note = 2'-O-methyl modified nucleotide
  modified_base 26: mod_base = OTHER; note = 2'-fluoro modified nucleotide
  modified_base 27: mod_base = OTHER; note = 2'-O-methyl modified nucleotide
  modified_base 28: mod_base = OTHER; note = 2'-fluoro modified nucleotide
  modified_base 29: mod_base = OTHER; note = 2'-O-methyl modified nucleotide
  modified_base 30: mod_base = OTHER; note = 2'-fluoro modified nucleotide
  modified_base 31: mod_base = OTHER; note = 2'-O-methyl modified nucleotide
  modified_base 32: mod_base = OTHER; note = 2'-fluoro modified nucleotide with phosphorothioate linkage
  modified_base 33: mod_base = OTHER; note = 2'-O-methyl modified nucleotide with phosphorothioate linkage
  modified_base 34: mod_base = OTHER; note = 2'-fluoro modified nucleotide with phosphorothioate linkage attached to trivalent N-acetylgalactosamine
tcaacaaggagtacccgggaagttgagaacaaat

SEQ ID NO: 166 - RNA, 33 nt, synthetic construct
  source 1..33: mol_type = other RNA
  modified_base 1: mod_base = OTHER; note = 5'-phosphate 2'-O-methyl modified nucleotide with phosphorothioate linkage
  modified_base 2: mod_base = OTHER; note = 2'-fluoro modified nucleotide with phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-O-methyl modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide with phosphorothioate linkage
  modified_base 15: mod_base = OTHER; note = 2'-O-methyl modified nucleotide with phosphorothioate linkage
  modified_base 16: mod_base = OTHER; note = 2'-fluoro modified nucleotide with phosphorothioate linkage
  modified_base 17: mod_base = OTHER; note = 2'-O-methyl modified nucleotide with phosphorothioate linkage
  modified_base 18: mod_base = OTHER; note = 2'-fluoro modified nucleotide with phosphorothioate linkage
  modified_base 19: mod_base = OTHER; note = 2'-O-methyl modified nucleotide with phosphorothioate linkage
  modified_base 20: mod_base = OTHER; note = 2'-O-methyl modified nucleotide
  modified_base 21: mod_base = OTHER; note = 2'-fluoro modified nucleotide
  modified_base 22: mod_base = OTHER; note = 2'-fluoro modified nucleotide
  modified_base 23: mod_base = OTHER; note = 2'-fluoro modified nucleotide
  modified_base 24: mod_base = OTHER; note = 2'-O-methyl modified nucleotide
  modified_base 25: mod_base = OTHER; note = 2'-fluoro modified nucleotide
  modified_base 26: mod_base = OTHER; note = 2'-O-methyl modified nucleotide
  modified_base 27: mod_base = OTHER; note = 2'-fluoro modified nucleotide
  modified_base 28: mod_base = OTHER; note = 2'-O-methyl modified nucleotide
  modified_base 29: mod_base = OTHER; note = 2'-fluoro modified nucleotide
  modified_base 30: mod_base = OTHER; note = 2'-O-methyl modified nucleotide
  modified_base 31: mod_base = OTHER; note = 2'-fluoro modified nucleotide with phosphorothioate linkage
  modified_base 32: mod_base = OTHER; note = 2'-O-methyl modified nucleotide with phosphorothioate linkage
  modified_base 33: mod_base = OTHER; note = 2'-fluoro modified nucleotide with phosphorothioate linkage attached to trivalent N-acetylgalactosamine
atagaagaaaaggtgggagaccttttcttctat

(19) United States
(12) Patent Application Publication
(10) Pub. No.: US 2026/0218180 A1
(43) Pub. Date: Jul. 30, 2026

(54) PRODUCTS AND COMPOSITIONS

(71) Applicant: SIRNAOMICS, INC., Germantown, MD (US)

(72) Inventors: Jack Wei, Germantown, MD (US); Dmitry Samarsky, Germantown, MD (US)

(73) Assignee: SIRNAOMICS, INC., Germantown, MD (US)

(21) Appl. No.: 19/142,520

(22) PCT Filed: Dec. 22, 2023
(86) PCT No.: PCT/US2023/085708

§ 371 Date: Jun. 23, 2025

Related U.S. Application Data
(60) Provisional application No. 63/434,688, filed on Dec. 22, 2022.

(51) Int. Cl. C12N 15/113; A61K 31/216; A61K 31/232; A61K 31/366; A61K 31/397; A61K 31/41; A61K 31/4178; A61K 31/4245; A61K 31/505; A61K 45/06

(52) U.S. Cl. CPC C12N 15/113; A61K 31/216; A61K 31/232; A61K 31/366; A61K 31/397; A61K 31/41; A61K 31/4178; A61K 31/4245; A61K 31/505; A61K 45/06; C12N 2310/14; C12N 2310/313; C12N 2310/315; C12N 2310/321; C12N 2310/322; C12N 2310/3231; C12N 2310/3233; C12N 2310/351; C12N 2310/3513; C12N 2310/3515

(57) ABSTRACT

The present disclosure relates to nucleic acid products that interfere with or inhibit APOC3 and AGT gene expression, together with methods for using the nucleic acids in the treatment of subjects.

### CROSS-REFERENCE TO RELATED APPLICATIONS

This application is a U.S. National Phase of PCT/US2023/085708 filed Dec. 22, 2023, which in turn claims priority to U.S. Provisional Patent Application Ser. No. 63/434,688, filed Dec. 22, 2022, the disclosure of which is hereby incorporated by reference in its entirety.

### SEQUENCE LISTING

The instant application contains a Sequence Listing submitted electronically in ST.26 (XML) format and hereby incorporated by reference in its entirety. The XML file, created on Dec. 6, 2023, is named 4690 00821 SL.xml and is 505,772 bytes in size.

### FIELD

The present disclosure relates to products, and compositions, and methods of their manufacture and use. In particular, the present disclosure relates to nucleic acid products that modulate, in particular interfere with or inhibit, APOC3 and AGT gene expression. Disclosed embodiments can therefore include methods, compounds, and compositions for reducing expression of APOC3 and AGT mRNA and protein in a human or an animal. Disclosed embodiments further include methods of treatment, prevention, and/or amelioraion of APOC3- and AGT-associated disorders.

### BACKGROUND

### APOC3

Triglycerides are the body's primary form of fat storage, providing energy to the body in times of need. Each triglyceride molecule consists of a glycerol backbone to which three fatty acids are esterified. Triglicerides can be taken in from the diet as such, or can be produced in the body from excess levels of carbohydrates and other nutrients and are transported in the blood to various tissues. In the aqueous environment of blood, however, triglycerides must be transported inside lipoproteins, spherical groups of molecules with an outer shell that protects the triglyceride cargo as it travels to tissues for storage or for breakdown to produce energy.

Hypertriglyceridemia (an elevated level of triglycerides in the blood), itself a recognized disorder is a causative or contributing factor in the development or progression of a range of disorders. For example, partly in conjunction with elevated levels of low-density lipoprotein (“LDL”) cholesterol, hypertriglyceridemia has been implicated in the development of atherosclerotic cardiovascular disease (“ASCVD”) and related metabolic and other disorders. A more comprehensive list of disorders associated with elevated levels of triglycerides is given in the embodiments disclosed further below.

Apolipoprotein C3 (“APOC3”) is secreted by the liver and the small intestine and is found on the surface of triglyceride-rich lipoproteins such as very low-density lipoproteins (“VLDL”) and chylomicrons. APOC3 is involved in the negative regulation of lipid catabolismespecially triglyceride catabolismand of the clearance from blood of VLDL, LDL and high-density lipoproteins (“HDL′). One of APOC3's functions is inhibiting lipoprotein lipase and hepatic lipase, enzymes involved in the breakdown of triglycerides to glycerol and free fatty acids, resulting in elevated levels of triglycerides. Dysregulation of APOC3 leading to a chronically elevated level in blood can lead to or signal the presence of disorders of the cardiovascular system, including the stimulation of vascular inflammation.

### AGT

The AGT gene encodes angiotensinogen, an upstream component of the renin-angiotensin-aldosterone system (RAAS), which regulates blood pressure and the balance of fluids and electrolytes in the body. Angiotensinogen is primarily secreted by the liver, and is a precursor to the angiotensin peptides, angiotensin I and angiotensin II. Angiotensinogen is converted by active renin to angiotensin I, which is then converted by angiotensin converting enzyme (ACE) to angiotensin II (Gribouval O, et al., Hum. Mutat. 2012, 33:316-26). Angiotensin II, a peptide hormone, causes blood vessels to narrow (vasoconstriction), which results in increased blood pressure. Angiotensin II also stimulates production of the hormone aldosterone, which triggers the absorption of salt and water by the kidneys. The increased amount of fluid in the body also increases blood pressure.

Hypertension and associated diseases may follow the dysregulation of RAAS and the expression of the AGT gene. Dysregulation of angiotensin II, in particular, excessive angiotensin II production, resulting in hypertension can lead to increased oxidative stress, promotion of inflammation, hypertrophy, and fibrosis in the heart, kidneys, and arteries, and may finally result in left ventricular fibrosis, arterial remodelling and glomerulosclerosis.

Hypertension is the most prevalent, controllable disease in developed countries, affecting 20-50% of adult populations. It is a major risk factor for a variety of diseases, disorders and conditions such aschronic kidney disease, stroke, myocardial infarction, heart failure, aneurysms (e.g. aortic aneurysm), peripheral artery disease, heart damage (e.g., heart enlargement or hypertrophy) and other cardiovascular related diseases, disorders, or conditions. In 2017, the guidelines for diagnosis, prevention, and treatment of hypertension were revised, promoting a further reduction in blood pressure to decrease risk of development of these conditions (see, e.g., Reboussin et al. Systematic Review for the 2017 ACC/AHA/AAPA/ABC/ACPM/AGS/APhA/ASH/ASPC/NMA/PCNA Guideline for the Prevention, Detection, Evaluation, and Management of High Blood Pressure in Adults: A Report of the American College of Cardiology/American Heart Association Task Force on Clinical Practice Guidelines. J Am Coll Cardiol. 2017 Nov. 7. pii: S0735-1097(17):41517-8. doi: 10.1016/j.jacc.2017.11.004; and Whelton et al. (Id. at 41519-1. doi: 10.1016/j jacc.2017.11.006).

Co-Occurrence of Disorders Resulting from Dysregulation of Expression of the APOC3 and AGT Genes

Hypertension and other AGT-associated disorders can also co-occur with APOC3-associated disorders such as dyslipidemia, more specifically, hypertriglyceridemia, hyperchylomicronaemia, and ASCVD.

### Treatment of Hypertriglyceridemia and Hypertension

Established treatments of hypertriglyceridemia include the administration of statins such as Rosuvastatin and Simvastatin as well as of fibrates, such as fenofibrate. Side effects and intolerance of statins make them inappropriate options for some patients.

Despite the large number of anti-hypertensive drugs available for treating hypertension, more than two-thirds of subjects require more than one anti-hypertensive agents selected from different drug classes. Side effects are increased with increasing numbers and types of medications and patient adherence to a medication regimen (and controlled blood pressure) are reduced as a result. Furthermore, several studies have suggested a potential relationship between chronic use of antihypertensive medications and deterioration in kidney function finding that antihypertensive agents to control blood pressure also impact kidney function independently of their effect on blood pressure (Tomlinson, et al (2013) PLOS ONE 8(11) Article ID e78465; The SPRINT Research Group (2015) AE7/1/373(22):2103-2116, ClinicalTrials.gov number, NCT01206062; Kidney Disease: Improving Global Outcomes (KDIGO) CKD Work Group (2013) Kidney International Supplements 3:1-150; Kamaroff, et al. (2018) (H indaw i International J C hr on Dis Article ID 1382705 | https://doi.org/10.1 155/2018/1382705). In addition to anti-hypertensive drugs, renal denervation, baroreceptor activation therapy, diet changes and lifestyle changes may reduce hypertension and reduce conditions associated with it (Paulis et al., Nat Rev Cardiol, 2012, 9:276-285).

Currently approved treatments for hypertension are limited, as a significant number of hypertensive patients do not achieve adequate blood pressure control. Drugs such as ACE inhibitors and angiotensin receptor blockers (ARBs) that target parts of the RAAS demonstrate limited effectiveness in the inhibition of the pathway (Nobakht et al., Nat Rev Nephrol, 2011, 7:356-359). Additionally, certain anti-hypertensive drugs such as ACE inhibitors are contraindicated due to their potential to compromise renal function in hypertensive patients with renal disease.

Therefore, there is a need to provide improved treatments with hypertriglyeridemia and/or hypertension and conditions that may develop.

### SUMMARY

The following aspects are non-limiting.

According to a first aspect, the present disclosure is directed to a nucleic acid construct comprising:
(a) a first nucleic acid portion that is at least partially complementary to at least a first portion of an RNA which is transcribed from an APOC3 gene;
(b) a second nucleic acid portion that is at least partially complementary to at least a second portion of an RNA which is transcribed from an AGT gene;
(c) a third nucleic acid portion that is at least partially complementary to said first nucleic acid portion of (a), so as to form a first nucleic acid duplex region therewith;
(d) a fourth nucleic acid portion that is at least partially complementary to said second nucleic acid portion of (b), so as to form a second nucleic acid duplex region therewith.

According to a second aspect, the present disclosure is directed to a composition comprising a nucleic acid construct according to the first aspect, and a physiologically acceptable excipient.

According to a third aspect, the present disclosure is directed to pharmaceutical composition comprising a nucleic acid construct according to the first aspect.

According to a fourth aspect, the present disclosure is directed to the nucleic acid construct according to the first aspect, for use in human or veterinary medicine or therapy.

According to a fifth aspect, the present disclosure is directed to a nucleic acid construct according to the first aspect for use in a method of treating, ameliorating and/or preventing a disease or disorder.

According to a sixth aspect, the present disclosure is directed to a method of treating a disease or disorder comprising administration of a nucleic acid construct according to the first aspect, to an individual in need of treatment.

According to a seventh aspect, the present disclosure is directed to a use of a nucleic acid construct according to the first aspect, for use in research as a gene function analysis tool.

According to an eighth aspect, the present disclosure is directed to a use of a nucleic acid construct according to the first aspect in the manufacture of a medicament for a treatment of a disease or disorder.

Advantageous and/or exemplary features of constructs according to the present disclosure are as follows:
1) they contain multiple (2 or more) at least partially double-stranded agents capable of triggering RNA interference, tied together into a single nanostructure predominantly through complementary (Watson-Crick) interactions;
2) optionally, other (e.g.) covalent bindings may be used to build the constructs and/or add various ligands (e.g. delivery/targeting moieties such as GalNAc and or other carbohydrates, cholesterol, peptides, or small molecules, optionally attached via linkers);
3) the constructs predominantly comprise chemically modified nucleotides (e.g. 2′F, 2′0Me, LNO, PNA, MOE, BNA, PMO, phosphorothioate, phosphodithioate, etc.), mostly (but not only) to increase resistance to nucleases;
4) the constructs contain “fragile” components (e.g. chemical linkers, unmodified nucleotides, etc.), which allow the constructs to disassemble upon exposure to certain biologic environments (e.g. exposure to extra- and/or intra-cellular fluids); particular examples could be (but not limited): a) cleavage of the oligo backbone by nucleases in the sites with non-modified nucleotides; b) cleavage of the chemical linkage due to the change of pH (e.g. in endosomes);
5) disassembly upon exposure to said certain biologic environments releases the components (e.g. said at least partially double-stranded agents capable of triggering RNA interference) to modulate (up- or down-regulate, advantageous down-regulate) target gene expression in cell s/organisms;
6) said constructs can be used to modulate, advantageously down-regulate or silence gene expression, to study gene function, or to treat various diseases associated with the target genes to be down-regulated.

### Effects Achieved by the Disclosed Nucleic Acid Constructs

The inventive nucleic acid constructs, including APOC3 targeting antisense strands and AGT targeting antisense strands, are capable of reducing APOC3 and AGT expression at the same time in an effective manner.

Furthermore, antisense strands which are shown to be active against APOC3 in form of an mxRNA (miniaturized short hairpins; for a more comprehensive definition see further below) perform surprisingly well when being part of the muRNA nucleic acid constructs (interfering RNAs comprising sequences targeting different target mRNAs and/or different regions of a given target mRNA; for a more comprehensive definition see further below) disclosed herein.

Advantageously, there may be certain differences between mxRNAs and muRNAs with regard to (i) sequence lengths, in particular of the sense portions, and (ii) modifications, modifications including 2′ modifications and modified phosphates.

As regards lengths, an advantageous design of mxRNAs is 14-5-14. This means a length of the antisense sequence of 14+5=19 nucleotides, wherein the 5 3′-terminal nucleotides constitute the loop in said mxRNA. Advantageously, the sense strand has a length of 14 nucleotides only. 14+5+14=33 nucleotides is the total length of such mxRNA.

Conversely, an advantageous design of muRNAs is 15-4-15. This means that on the first strand of said muRNA, there is an antisense sequence directed to a region of a first target mRNA which has a length of 15+4=19 nucleotides, and, directly attached thereto, a sense sequence which identical to a region of a second target mRNA or a different region of the same target mRNA, said sense sequence advantageously having a length of 15 nucleotides. The same applies, mutatis mutandis, to the second strand of said muRNA which comprises an antisense sequence directed to a second mRNA target or a different region of the same target mRNA and furthermore a sense sequence which is complementary to the antisense sequence on the first strand of said muRNA. In terms of total length, each strand of such advantageous muRNA has a length of 15+4+15=34 nucleotides.

As a consequence, if mxRNA antisense and sense sequences are to be employed in the context of a muRNA, the antisense sequence can be used without modifications of its nucleobase sequence. As regards the sense sequence, and given that 14 nucleotide sense sequences are advantageous in mxRNAs, one further nucleotide is advantageously added to the sense sequence when using an mxRNA as a basis for designing a 15-4-15 muRNA. In particular, the nucleobase at the additional position 15 has to be added at the 5′ terminus of said sense sequence and has to be complementary to the nucleobase at position 15 of the antisense sequence directed to the same target mRNA or region thereof. Positions are always counted in 5′ to 3′ direction.

As regards modifications, the following is of note. Modifications of the 2′ positions are a means to stabilize compounds of the presently disclosed embodiments (including muRNAs) as well as mxRNAs (which may serve as a basis to design muRNAs). The same applies to phosphorothioates which are a means to stabilize as well, in particular in regions of both mxRNAs and muRNAs which are not engaged in base pairing or are located close to or at the termini of the respective strand(s). At the same time it is advantageous for the design of muRNAs that they disassamble in a cellullar environment to give rise to two distinct short RNAs which are capable of being loaded separately into the RISC complex. An advantageous location within said muRNAs which allows for such disassembly is position 19 of the respective antisense sequence. Therefore, when an mxRNA is used as a basis for the design of a muRNA, and to the extent the nucleoside at position 19 of the antisense sequence of said mxRNA would be a 2′ modified nucleoside, said modification may be dispensed with to give rise to a 2′ unmodified nucleoside which carries an OH group at the 2′ position. Otherwise, it is advantageous for the muRNAs of the disclosed embodiments to use an alternating pattern of 2′-F and 2′-0Me modifications beginning with a 2′-0Me modification at the 5′ end.

As regards phosphorothiotes, these advantageously occur at the first two internucleoside linkages at either terminus of any strand and furthermore between nucleosides in a loop (in case of mxRNAs) or bulge (in case of muRNAs). As such, phosphorothioates may be used for connecting the nucleosides at positions 14 to 19 of an mxRNA, amounting to a total of six phosphorothioates in the loop of an mxRNA design as disclosed above. Since particularly advantageous muRNAs follow the abovementioned 15-4-15 design, the location of phosphorothioates in the central region of each strand of said muRNAs may not be the same as in the loop of parent mxRNAs which advantageously follow a 14-5-14 design. In particular, advantageous positions of phosphorothioates in the central part of muRNAs are at positions 15 to 18 of each strand of said muRNA. Since muRNAs will be cleaved upon administration, the newly (yet to be) formed termini of the cleavage products are advantageously protected as well. This is why not only nucleosides in the bulge of an muRNA (positions 15 to 18), but also the two nucleosides following the fragile nucleotide at position 19 (said two nucleosides being at positions 20 and 21) are linked by phosphorothioates.

When referring to positions of phosphorothioates, it is understood that a phosphorothioate at position n refers to said phosphorothioate connecting nucleosides at positions n and n+1, as a 3′ to 5′ connection.

To conclude, while components of mxRNAs can be used in a muRNA context, it is advantageous to perform the above-described adjustments. As shown in the Examples, such adjustments do not impair activity. As a consequence, performance of an antisense region within an mxRNA context is surprisingly predictive of its performance in an muRNA context.

Altogether, the nucleic acid constructs according to the present disclosure are capable of addressing the technical problem set forth above. Evidence can be found in the Examples.

Furthermore, it was surprisingly found that the mentioned effects are achieved by using oligomeric compounds according to the disclosed embodiments for inhibiting the expression of APOC3 and AGT genes in the form of muRNA constructs having a reduced number of nucleosides, e.g. 34 nucleosides on each strand, compared to conventional siRNA molecules having a greater total number of nucleosides when taking into account that two conventional siRNA would have to employed for the purpose of inhibiting two target genes. This difference can e.g. make a synthesis of muRNA molecules more cost and production efficient, because less units are needed.

For certain oligomeric compounds in the form of muRNA constructs for inhibiting the expression of APOC3 and AGT genes, it was surprisingly found out that the aforementioned effects can be achieved by using short sense strands within the muRNA having a length of 14 or 15 nucleosides which is shorter than the length of the sense strands in conventional siRNA molecules.

The effects and technical advantages achieved by using the inventive oligomeric compounds for inhibiting APOC3 and AGT expression will become apparent in more detail in the detailed description and the examples.

### BRIEF DESCRIPTION OF THE DR WINGS

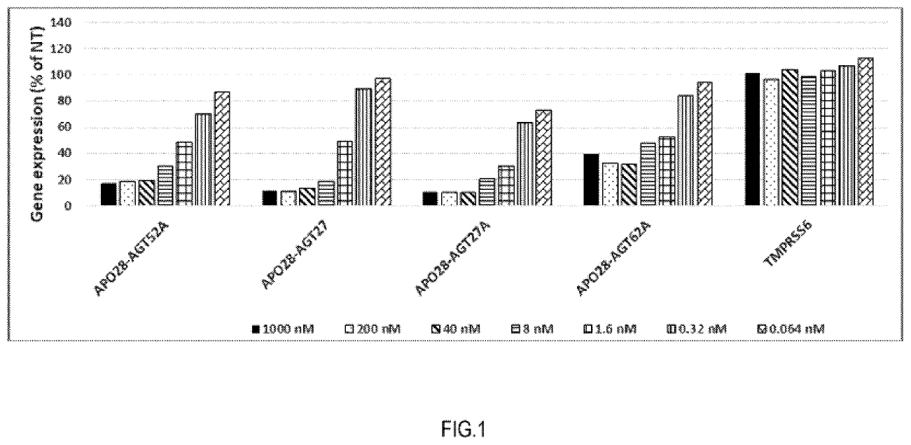

FIG. 1 shows concentration dependence of AGT inhibition in vitro by constructs of the disclosure, and by a construct targeting an unrelated mRNA (TMPRSS6; neagtive control).

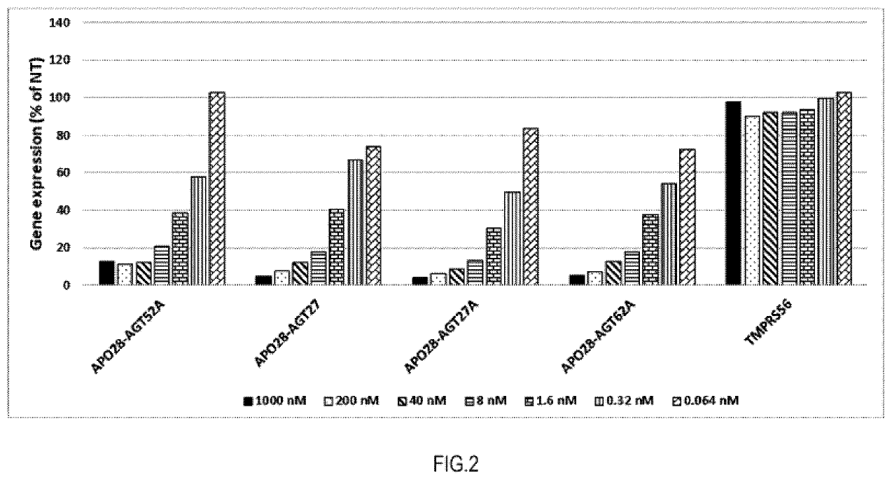

FIG. 2 shows concentration dependence of APOC3 inhibition in vitro Fby constructs of the disclosure, and by a construct targeting an unrelated mRNA (TMPRSS6; negative control).

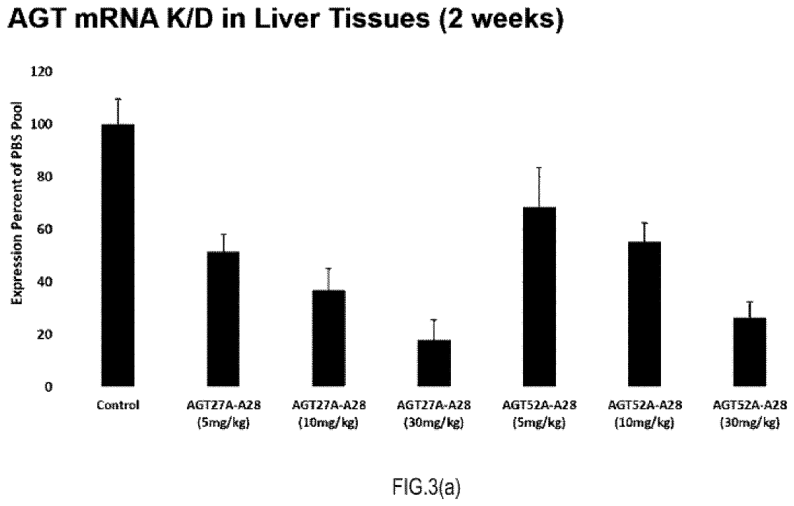

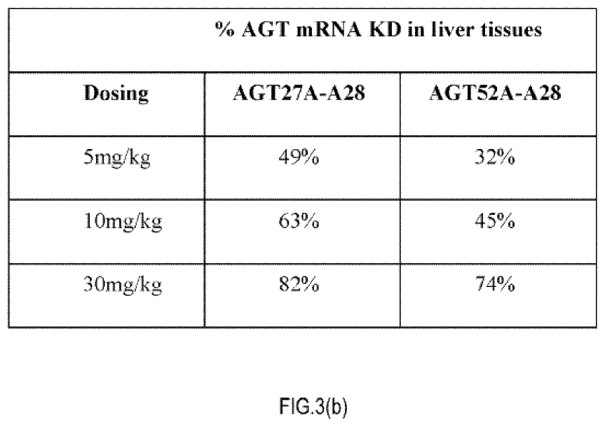

FIG. 3 (a) shows AGT mRNA knockdown in liver tissue by constructs of the disclosure and illustrates an expression as percent of PBS pool, by dose;

FIG. 3b shows AGT mRNA knockdown in liver tissue by constructs of the disclosure and illustrates AGT mRNA KD in liver tissue, by dose.

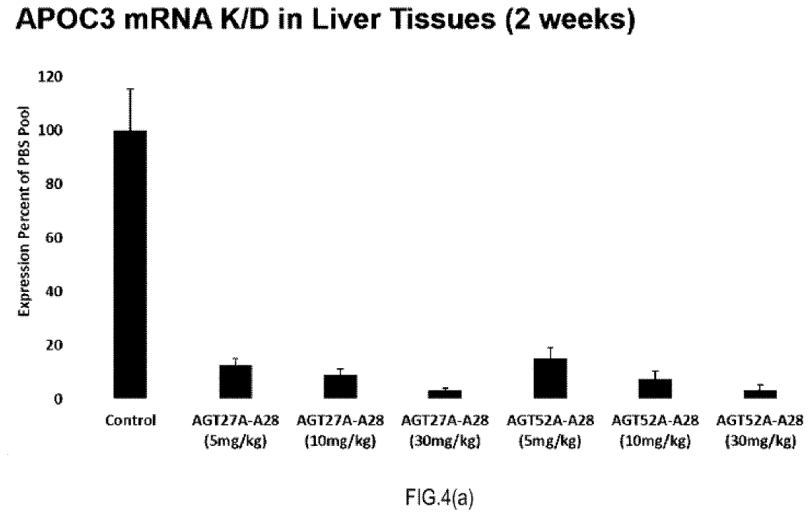

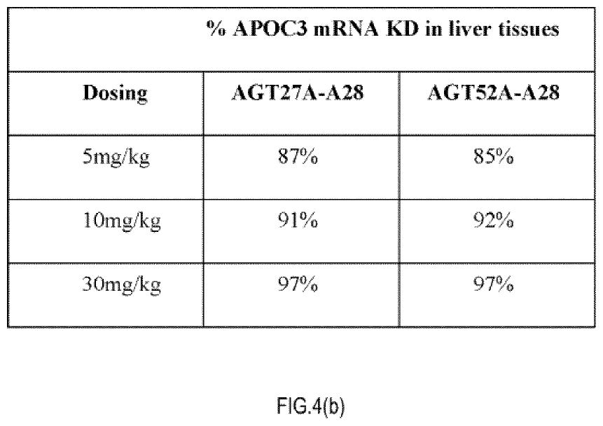

FIG. 4 (a) shows APOC3 mRNA knockdown in liver tissue by constructs of the disclosure and illustrates an expression as percent of PBS pool, by dose;

FIG. 4b shows APOC3 mRNA knockdown in liver tissue by constructs of the disclosure and illustrates an APOC3 mRNA KD in liver tissue, by dose.

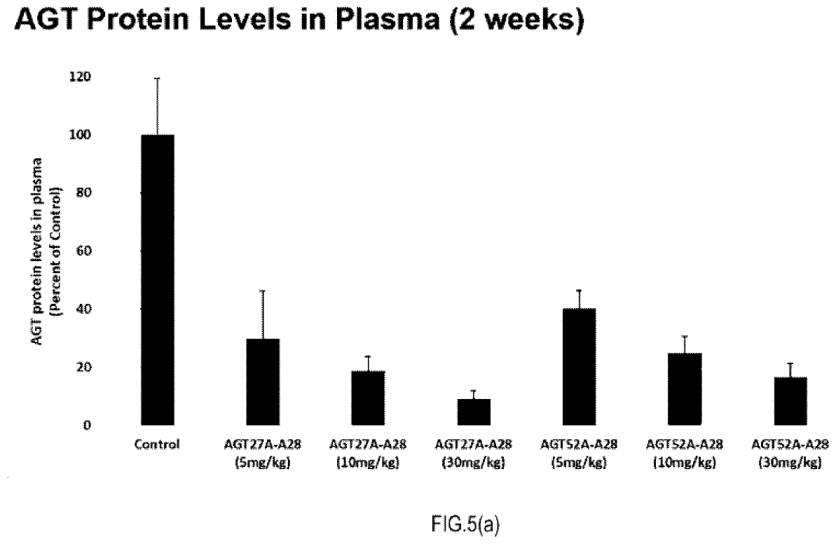

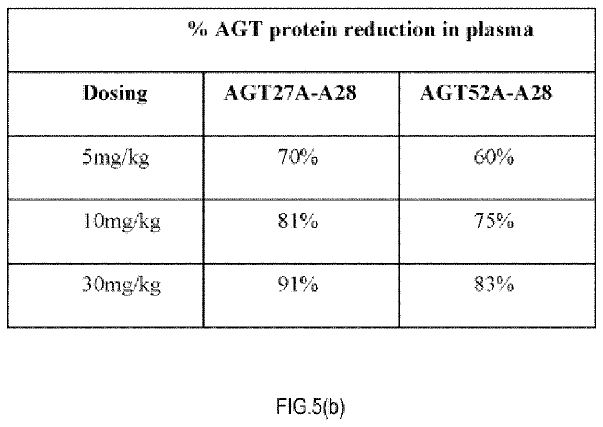

FIG. 5 (a) shows AGT protein knockdown in plasma by constructs of the disclosure and illustrates an AGT protein levels in plasma (% of control);

FIG. 5b shows AGT protein knockdown in plasma by constructs of the disclosure and illustrates an AGT protein reduction in plasma, by dose.

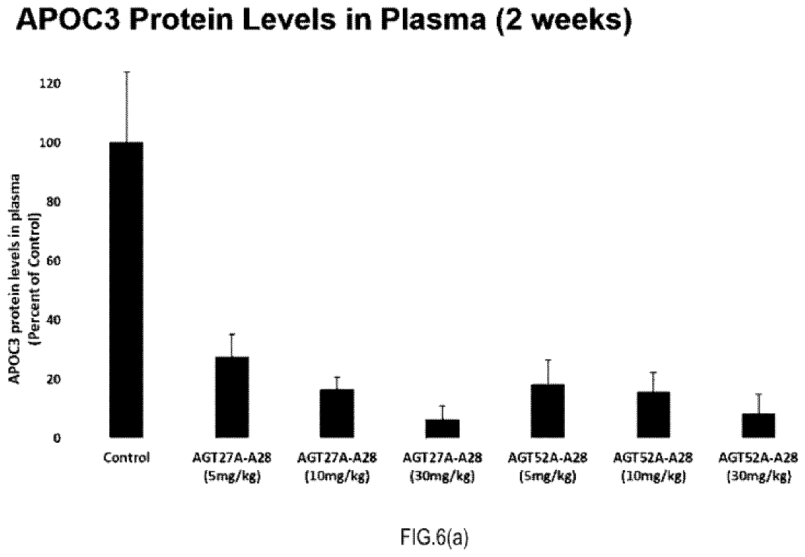

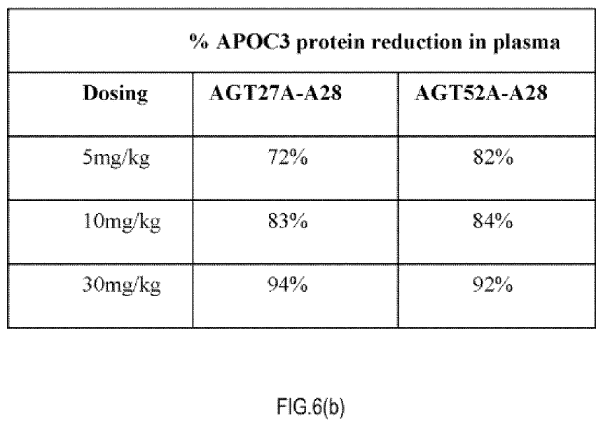

FIG. 6 (a) shows APOC3 protein knockdown in plasma by constructs of the disclosure and illustrates APOC3 protein levels in plasma (% of control);

FIG. 6b shows APOC3 protein knockdown in plasma by constructs of the disclosure and illustrates APOC3 protein reduction in plasma, by dose.

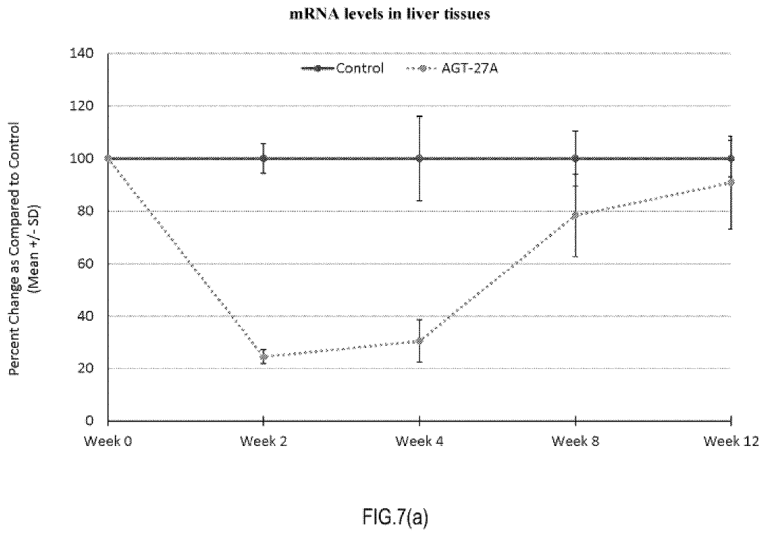

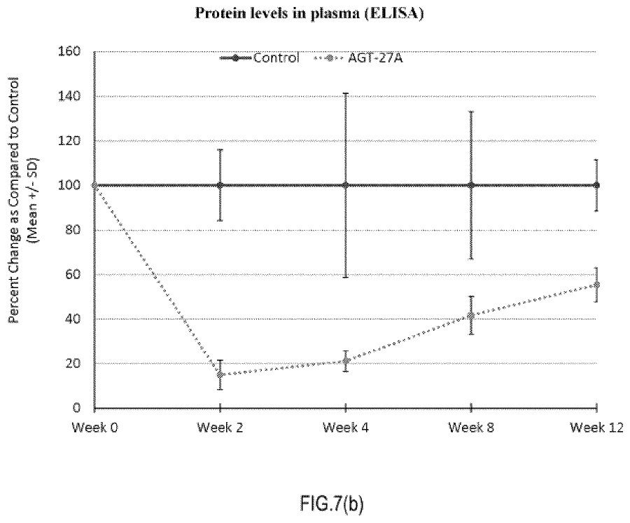

FIG. 7 (a) shows the mRNA levels in liver tissues (AGT-27A versus controls). Duration response: week 2: 75% NADIR; week 4: 70% mRNA KD; weeks 8 (21%) and 12 (9%), return to control levels.

FIG. 7 (b) shows protein levels in plasma using ELISA. Duration response: week 2: 85% maximum reduction; week 4: 79% reduction; week 8: 58% reduction; and week 12: 44% reduction.

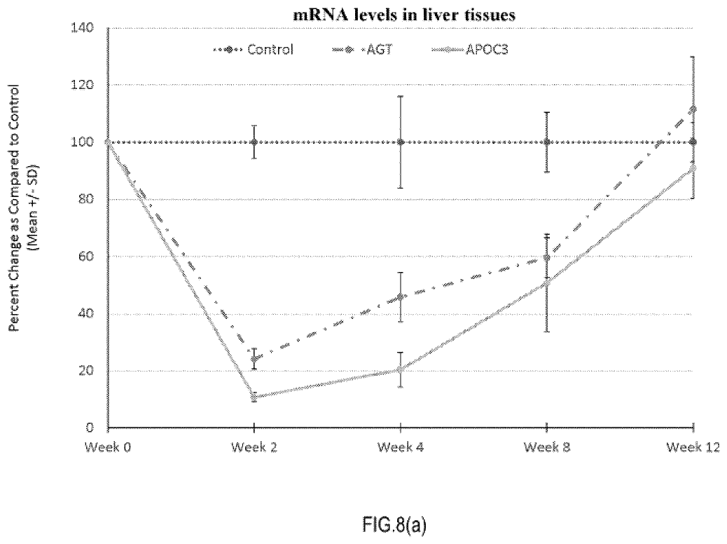

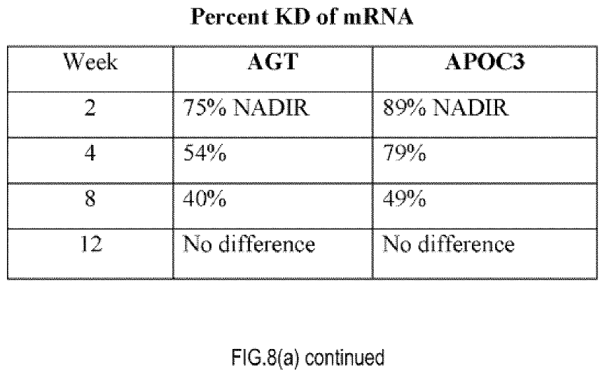

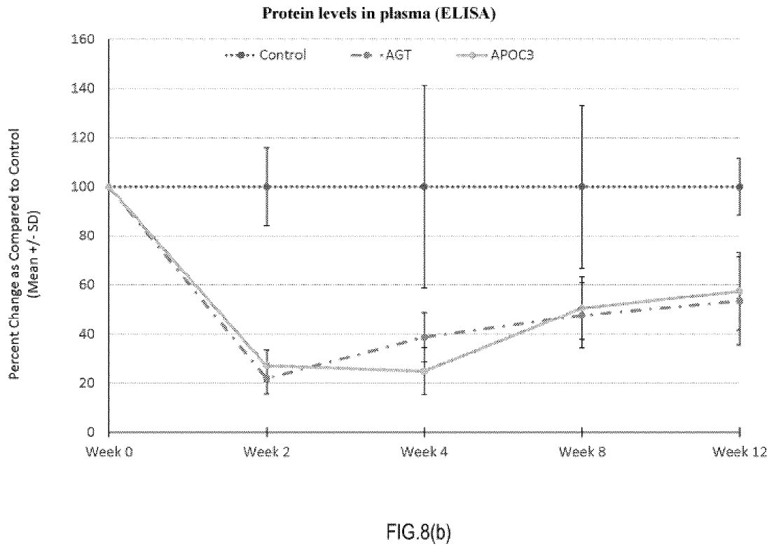

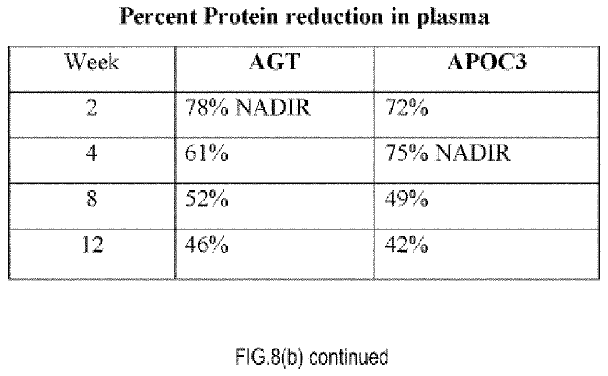

FIG. 8 (a) shows the mRNA levels in liver tissues (AGT and APOC3 versus controls); the percent KD of mRNA is shown at weeks 2, 4, 8 and 12 in the table following the line graph.

FIG. 8 (b) shows the protein levels in plasma using ELISA (AGT and APOC3 versus controls); the protein reduction in plasma is shown at weeks 2, 4, 8 and 12 in the table following the line graph.

### DETAILED DESCRIPTION

Provided are embodiments for siRNA molecules, pharmaceutical compositions and methods of making them, their administration, delivery and use in treatment of AGT- and APCO3-realted diseases and disorders.

Further implementations of the disclosed embodiments are described below by way of example only. These examples represent the advantageous ways of putting the disclosure into practice that are currently known to the applicant although they are not the only ways in which this could be achieved.

For the sake of clarity, it is noted here that implementations or embodiments labelled “advantageous” or “advantageously” are not intended to limit the scope of the claims but to show optional embodiments.

Features of different aspects and implementations or embodiments may be combined as appropriate, as would be apparent to a skilled person.

### Definitions

The following definitions apply to the entire disclosure. In many instances, the definitions, in addition to the respective definition as such, provide non-exhaustive listings of possible, optional or advantageous implementations.

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21st edition, 2005; and “Antisense Drug Technology, Principles, Strategies, and Applications” Edited by Stanley T. Crooke, CRC Press, Boca Raton, Florida; and Sambrook et al., “Molecular Cloning, A laboratory Manual,” 2nd Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “excipient” means any compound or mixture of compounds that is added to a composition as provided herein that is suitable for delivery of an oligomeric compound.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety, phosphate-linked nucleosides also being referred to as “nucleotides”. The structural features and/or the lengths of oligomeric compounds or nucleic acid constructs disclosed herein is expressed in terms of “nucleosides” or “nucleotides”.

As used herein, “chemical modification” or “chemically modified” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA. A “naturally occurring sugar moiety” as referred to herein is also termed as an “unmodified sugar moiety”. In particular, such a “naturally occurring sugar moiety” or an “unmodified sugar moiety” as referred to herein has a —H (DNA sugar moiety) or —OH (RNA sugar moiety) at the 2′-position of the sugar moiety, especially a —H (DNA sugar moiety) at the 2′-position of the sugar moiety.

As used herein, “sugar moiety” means a naturally occurring sugar moiety or a modified sugar moiety of a nucleoside. As used herein, “modified sugar moiety,” means a substituted sugar moiety or a sugar surrogate.

As used herein, “substituted sugar moiety” means a furanosyl that has been substituted. Substituted sugar moieties include, but are not limited to, furanosyls comprising substituents at the 2′-position, the 3′-position, the 5′-position and/or the 4′-position. Certain substituted sugar moieties are bicyclic sugar moieties.

As used herein, “2′-substituted sugar moiety” means a furanosyl comprising a substituent at the 2′-position other than H or OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring).

As used herein, “MOE” means —OCH₂CH₂OCH₃.

As used herein, “2′-F nucleoside” refers to a nucleoside comprising a sugar comprising fluorine at the 2′ position. Unless otherwise indicated, the fluorine in a 2′-F nucleoside is in the ribo position (replacing the OH of a natural ribose). Duplexes of uniformly modified 2′-fluorinated (ribo) oligonucleotides hybridized to RNA strands are not RNase H substrates while the analogues retain RNase H activity.

As used herein the term “sugar surrogate” means a structure that does not comprise a furanosyl and that can replace the naturally occurring sugar moiety of a nucleoside, such that the resulting nucleoside sub-units are capable of linking together and/or linking to other nucleosides to form an oligomeric compound which is capable of hybridizing to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g, 6-membered carbocyclic bicyclic sugar surrogates optionally comprising additional substituents). Sugar surrogates also include more complex sugar replacements (e.g, the non-ring systems of peptide nucleic acid). Sugar surrogates include without limitation morpholinos, cyclohexenyls and cyclohexitols.

As used herein, “bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments, the 4 to 7 membered ring is a furanosyl. In certain such embodiments, the bridge connects the 2 ‘-carbon and the 4’-carbon of the furanosyl.

As used herein, “nucleotide” means a nucleoside further comprising a phosphate linking group. As used herein, “linked nucleosides” may or may not be linked by phosphate linkages and thus includes, but is not limited to, “linked nucleotides.” As used herein, “linked nucleosides” are nucleosides that are connected in a continuous sequence (i.e., no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding, more specifically hydrogen bonding, with a complementary naturally occurring nucleobase of another oligonucleotide or nucleic acid. Nucleobases may be naturally occurring or may be modified.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase,” means any nucleobase that is not a naturally occurring nucleobase.

As used herein, “modified nucleoside” means a nucleoside comprising at least one chemical modification compared to naturally occurring RNA or DNA nucleosides. Modified nucleosides can comprise a modified sugar moiety and/or a modified nucleobase.

As used herein, “bicyclic nucleoside” or “BNA” means a nucleoside comprising a bicyclic sugar moiety.

As used herein, “locked nucleic acid nucleoside” or “LNA” means a nucleoside comprising a bicyclic sugar moiety comprising a 4′-CH₂—O-2′bridge.

As used herein, “2 ‘-substituted nucleoside” means a nucleoside comprising a substituent at the 2’-position of the sugar moiety other than H or OH. Unless otherwise indicated, a 2 substituted nucleoside is not a bicyclic nucleoside.

As used herein, “deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

As used herein, “modified oligonucleotide” means an oligonucleotide comprising at least one modified nucleoside and/or at least one modified internucleoside linkage.

Advantageously modified internucleoside linkages are those, which confer increased stability as compared to the naturally occurring phosphodiesters. “Stability” refers in particular to stability against hydrolysis including enzyme-catalyzed hydrolysis, enzymes including exonucleases and endonucleases.

Advantageous positions for such modified internucleoside linkages include the termini and the hairpin loop of single-stranded oligomeric compounds of the disclosed embodiments. For example, the internucleoside linkages connecting first and second nucleoside and second and third nucleoside counting from the 5′ terminus, and/or the internucleoside linkages connecting first and second nucleoside and second and third nucleoside counting from the 3′ terminus are modified. In addition, a linkage connecting the terminal nucleoside of the 3′ terminus with a ligand, such as GalNAc, may be modified.

As discussed above, advantageous positions are in the hairpin loop of said singlestranded oligomeric compounds. In particular, all linkages, all but one linkages or the majority of linkages in the hairpin loop are modified. As used herein, “linkages in the hairpin loop” designates the linkages between nucleosides, which are not engaged in base pairing. For example, in a hairpin loop consisting of five nucleosides, there are four linkages between nucleosides which are not engaged in base pairing. Advantageously, the term “linkages in the hairpin loop” also extends to the linkages connecting the stem to the loop, i.e., those linkages which connect a base-paired nucleoside to a non-based paired nucleoside. Generally, there are two such positions in hairpins and mxRNAs in accordance with the disclosed embodiments.

Most advantageous is that modified internucleoside linkages are at both termini and in the hairpin loop.

As used herein, “linkage” or “linking group” means a group of atoms that link together two or more other groups of atoms.

As used herein “internucleoside linkage” means a covalent linkage between adjacent nucleosides in an oligonucleotide.

As used herein “naturally occurring internucleoside linkage” means a 3′ to 5′ phosphodiester linkage.

As used herein, “modified internucleoside linkage,” means any internucleoside linkage other than a naturally occurring internucleoside linkage. In particular, a “modified internucleoside linkage” as referred to herein can include a modified phosphorous linking group such as a phosphorothioate or phosphorodithioate internucleoside linkage.

As used herein, “terminal internucleoside linkage” means the linkage between the last two nucleosides of an oligonucleotide or defined region thereof.

As used herein, “phosphorus linking group” means a linking group comprising a phosphorus atom and can include naturally occurring phosphorous linking groups as present in naturally occurring RNA or DNA, such as phosphodiester linking groups, or modified phosphorous linking groups that are not generally present in naturally occurring RNA or DNA, such as phosphorothioate or phosphorodithioate linking groups. Phosphorus linking groups can therefore include without limitation, phosphodiester, phosphorothioate, phosphorodithioate, phosphonate, methylphosphonate, phosphoramidate, phosphorothioamidate, thionoalkylphosphonate, phosphotriesters, thionoalkylphosphotriester and boranophosphate.

As used herein, “internucleoside phosphorus linking group” means a phosphorus linking group that directly links two nucleosides.

As used herein, “oligomeric compound” means a polymeric structure comprising two or more substructures. In certain embodiments, an oligomeric compound comprises an oligonucleotide, such as a modified oligonucleotide. In certain embodiments, an oligomeric compound further comprises one or more conjugate groups and/or terminal groups and/or ligands. In certain embodiments, an oligomeric compound consists of an oligonucleotide. In certain embodiments, an oligomeric compound comprises a backbone of one or more linked monomeric sugar moieties, where each linked monomeric sugar moiety is directly or indirectly attached to a heterocyclic base moiety. In certain embodiments, oligomeric compounds may also include monomeric sugar moieties that are not linked to a heterocyclic base moiety, thereby providing abasic sites. Oligomeric compounds may be defined in terms of a nucleobase sequence only, i.e., by specifying the sequence of A, G, C, U (or T). In such a case, the structure of the sugar-phosphate backbone is not particularly limited and may or may not comprise modified sugars and/or modified phosphates. On the other hand, oligomeric compounds may be more comprehensively defined, i.e., by specifying not only the nucleobase sequence, but also the structure of the backbone, in particular the modification status of the sugars (unmodified, 2′-0Me modified, 2′-F modified etc.) and/or of the phosphates. An mxRNA is one non-limiting example for an oligomeric compound.

As used herein, “nucleic acid construct” or “construct” refers to an assembly of two or more, such as four oligomeric compounds, said compounds being referred to as “portions” in the context of the first aspect of the disclosed embodiments. Said oligomeric compounds may be connected to each other by covalent bonds such phosphodiester bonds as they occur in naturally occurring nucleic acids or modified versions thereof as disclosed herein, and/or by non-covalent bonds such as hydrogen bonds, advantageous hydrogen bonds between nucleobases such as Watson-Crick base pairing. In certain embodiments, advantageous is that a construct comprises four oligomeric compounds, wherein a first and a fourth compound or portion as well as a second and third compound or portion are connected covalently, respectively, thereby giving rise to two nucleic acid strands which nucleic acid strands are bound to each other by hydrogen bonds. Owing to the covalent connection, what results is, strictly speaking, two compounds which are the two strands. Complementarity between said strands may be throughout, but is not necessarily so. In particular, exemplary embodiments provide for an antisense region targeting an APOC3 mRNA to be connected covalently with a sense region which is identical to a region of an AGT mRNA, and of an antisense region complementary to said sense region to be connected covalently to a sense region which is complementary to said an antisense region targeting an APOC3 mRNA. Since antisense and sense regions of the parent single-target-directed RNA molecules do not need to have the same length and advantageously do not have the same length with antisense portions being longer than sense portions, an advantageous construct of the disclosed embodiments contains a central region where the 3′ regions of the antisense portions of the parent single-target-directed RNA molecules face each other. In that region generally no or only partial base pairing will occur, while full complementarity is not excluded. Otherwise, where antisense and sense portions of the respective parent RNA molecules face each other; there is complementarity, advantageously full complementarity or 1 or 2 mismatches. A muRNA is a non-limiting example for a nucleic acid construct.

The term “strand” has its art-established meaning and refers to a plurality of linked nucleosides, the linker not being particularly limited, but including phosphodiesters and variants thereof as disclosed herein. A strand may also be viewed as a plurality of linked nucleotides in which case the linker would be a covalent bond.

As used herein, “terminal group” means one or more atom attached to either, or both, the 3′ end or the 5′ end, also called “terminus” of an oligonucleotide. In certain embodiments, a terminal group comprises one or more terminal group nucleosides, whereas a “terminal nucleoside” is only one nucleotide at the respective end (5′ end or 3′ end).

As used herein, “conjugate” or “conjugate group” means an atom or group of atoms bound to an oligonucleotide or oligomeric compound. In certain embodiments, a conjugate group links a ligand to a modified oligonucleotide or oligomeric compound. In general, conjugate groups can modify one or more properties of the compound to which they are attached, including, but not limited to pharmacodynamic, pharmacokinetic, binding, absorption, cellular distribution, cellular uptake, charge and/or clearance properties.

As used herein, “conjugate linker” or “linker” in the context of a conjugate group means a portion of a conjugate group comprising any atom or group of atoms and which covalently link an oligonucleotide to another portion of the conjugate group. In certain embodiments, the point of attachment on the oligomeric compound is the 3 ‘-oxygen atom of the 3’-hydroxyl group of the 3′ terminal nucleoside of the oligonucleotide. In certain embodiments, the point of attachment on the oligomeric compound is the 5′-oxygen atom of the 5′-hydroxyl group of the 5′ terminal nucleoside of the oligonucleotide. In certain embodiments, the bond for forming attachment to the oligomeric compound is a cleavable bond. In certain such embodiments, such cleavable bond constitutes all or part of a cleavable moiety.

In certain embodiments, conjugate groups comprise a cleavable moiety (e.g., a cleavable bond or cleavable nucleoside) and ligand portion that can comprise one or more ligands, such as a carbohydrate cluster portion, such as an N-Acetyl-Galactosamine, also referred to as “GalNAc”, cluster portion. In certain embodiments, the carbohydrate cluster portion is identified by the number and identity of the ligand. For example, in certain embodiments, the carbohydrate cluster portion comprises 2 GalNAc groups. For example, in certain embodiments, the carbohydrate cluster portion comprises 3 GalNAc groups and this is particularly advantageous. In certain embodiments, the carbohydrate cluster portion comprises 4 GalNAc groups. Such ligand portions are attached to an oligomeric compound via a cleavable moiety, such as a cleavable bond or cleavable nucleoside. The ligands can be arranged in a linear or branched configuration, such as a biantennary or triantennary configurations. An advantageous carbohydrate cluster has the following formula:

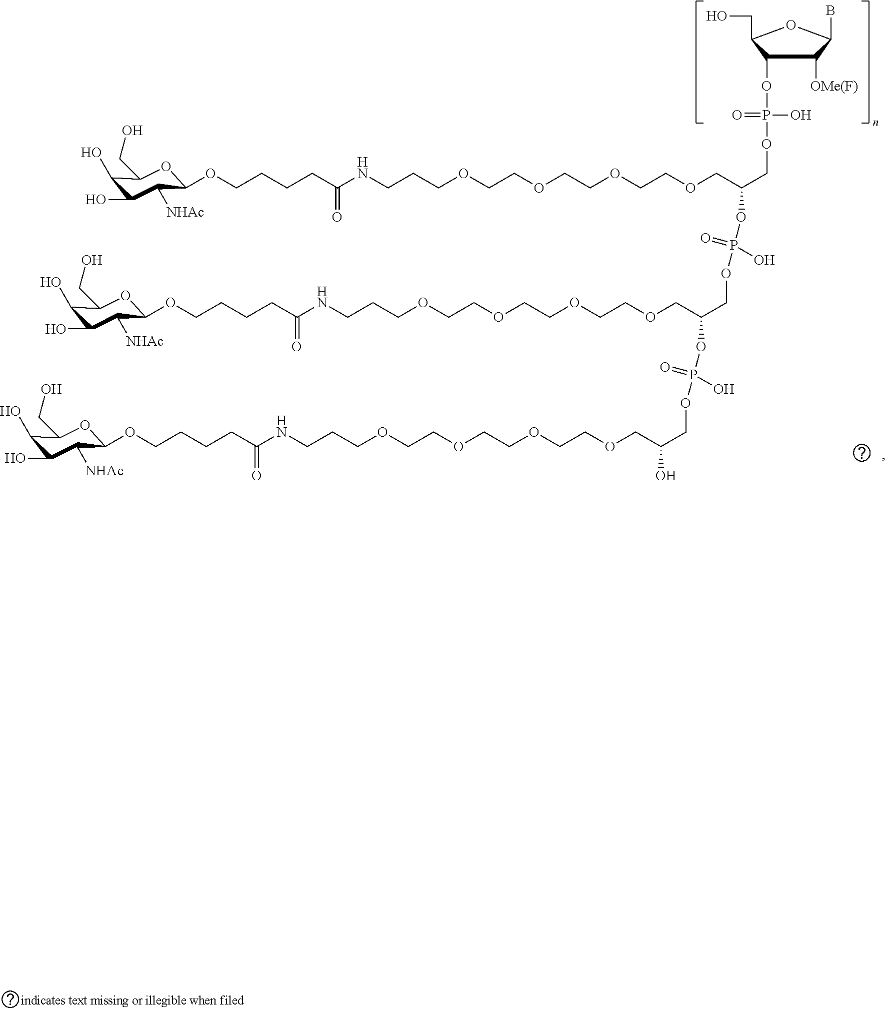

wherein in said structural formula one, two, or three phosphodiester linkages can also be substituted by phosphorothioate linkages.

As used herein, “cleavable moiety” means a bond or group that is capable of being cleaved under physiological conditions. In certain embodiments, a cleavable moiety is cleaved inside a cell or sub-cellular compartments, such as an endosome or lysosome. In certain embodiments, a cleavable moiety is cleaved by endogenous enzymes, such as nucleases. In certain embodiments, a cleavable moiety comprises a group of atoms having one, two, three, four, or more than four cleavable bonds. In certain embodiments, a cleavable moiety is a phosphodiester linkage.

As used herein, “cleavable bond” means any chemical bond capable of being broken.

As used herein, “carbohydrate cluster” means a compound having one or more carbohydrate residues attached to a linker group.

As used herein, “modified carbohydrate” means any carbohydrate having one or more chemical modifications relative to naturally occurring carbohydrates.

As used herein, “carbohydrate derivative” means any compound which may be synthesized using a carbohydrate as a starting material or intermediate.

As used herein, “carbohydrate” means a naturally occurring carbohydrate, a modified carbohydrate, or a carbohydrate derivative. A carbohydrate is a biomolecule including carbon (C), hydrogen (H) and oxygen (O) atoms. Carbohydrates can include monosaccharide, di saccharides, trisaccharides, tetrasaccharides, oligosaccharides or polysaccharides, such as one or more galactose moieties, one or more lactose moieties, one or more N-Acetyl-Galactosamine moieties, and/or one or more mannose moieties. A particularly advantageous carbohydrate is N-Acetyl-Galactosamine.

As used herein, “strand” means an oligomeric compound comprising linked nucleosides.

As used herein, “single strand” or “single-stranded” means an oligomeric compound comprising linked nucleosides that are connected in a continuous sequence without a break there between. Such single strands may include regions of sufficient self-complementarity so as to be capable of forming a stable self-duplex in a hairpin structure.

As used herein, “hairpin” means a single stranded oligomeric compound that includes a duplex formed by base pairing between sequences in the strand that are self-complementary and opposite in directionality.

As used herein, “hairpin loop” means an unpaired loop of linked nucleosides in a hairpin that is created by hybridization of the self-complementary sequences. The resulting structure looks like a loop or a U-shape.

In particular, short hairpin RNA, also denoted as shRNA, comprises a duplex region and a loop connecting the regions forming the duplex. The end of the duplex region, which does not carry the loop, may be blunt-ended or carry (a) 3′ and/or (a) 5′ overhang(s). Preference is given to blunt-ended constructs. The term “shRNA” is more generic than “mxRNA”, as defined below, and may include compounds in which the loop is not or not exclusively formed by a part of an antisense strand. In particular, shRNA includes an antisense strand, also called a guide strand, being complementary to a region of a target RNA, and a sense strand, i.e., a passenger strand, being substantially complementary to said antisense strand. More particularly, the antisense strand and the sense strand within the shRNA are directly linked, e.g. by a phosphate or a phosphorothioate, or linked by a third portion of linked nucleosides forming the loop, which means that the 3′ end of the antisense strand is linked to the 5′ end of the sense strand via covalent bonding over several other groups. Such direct linkage does not include a gap or nick.

As used herein, “directionality” means the end-to-end chemical orientation of an oligonucleotide based on the chemical convention of numbering of carbon atoms in the sugar moiety meaning that there will be a 5 ‘-end defined by the 5’ carbon of the sugar moiety, and a 3′-end defined by the 3′ carbon of the sugar moiety. In a duplex or double stranded oligonucleotide, the respective strands run in opposite 5′ to 3′ directions to permit base pairing between them.

As used herein, “duplex”, or also abbreviated as “dup”, means two or more complementary strand regions, or strands, of an oligonucleotide or oligonucleotides, hybridized together by way of non-covalent, sequence-specific interaction there between. Most commonly, the hybridization in the duplex will be between nucleobases adenine (A) and thymine (T), and/or (A) adenine and uracil (U), and/or guanine (G) and cytosine (C). The duplex may be part of a single stranded structure, wherein self-complementarity leads to hybridization, or as a result of hybridization between respective strands in a double stranded construct.

As used herein, “double strand” or “double stranded” means a pair of oligomeric compounds that are hybridized to one another. In certain embodiments, a double-stranded oligomeric compound comprises a first and a second oligomeric compound.

As used herein, “expression” means the process by which a gene ultimately results in a protein. Expression includes, but is not limited to, transcription, post-transcriptional modification (e.g, splicing, polyadenylation, addition of 5-cap), and translation.

As used herein, “transcription” or “transcribed” refers to the first of several steps of DNA based gene expression in which a target sequence of DNA is copied into RNA (especially mRNA) by the enzyme RNA polymerase. During transcription, a DNA sequence is read by an RNA polymerase, which produces a complementary, antiparallel RNA sequence called a primary transcript.

As used herein, “target sequence” means a sequence to which an oligomeric compound is intended to hybridize to result in a desired activity with respect to APOC3 or AGT expression. Oligonucleotides have sufficient complementarity to their target sequences to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In both DNA and RNA, guanine (G) is complementary to cytosine (C). In certain embodiments, complementary nucleobase means a nucleobase of an oligomeric compound that is capable of base pairing with a nucleobase of its target sequence. For example, if a nucleobase at a certain position of an oligomeric compound is capable of hydrogen bonding with a nucleobase at a certain position of a target sequence, then the position of hydrogen bonding between the oligomeric compound and the target sequence is considered complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides) means the capacity of such oligomeric compounds or regions thereof to hybridize to a target sequence, or to a region of the oligomeric compound itself, through nucleobase complementarity.

Complementary oligomeric compounds need not have nucleobase complementarity at each nucleoside. Rather, some mismatches are tolerated. In certain embodiments, complementary oligomeric compounds or regions are complementary at least at 70% of the nucleobases (at least 70% complementary). In certain embodiments, complementary oligomeric compounds or regions are at least 80% complementary. In certain embodiments, complementary oligomeric compounds or regions are at least 90% complementary. In certain embodiments, complementary oligomeric compounds or regions are at least 95% complementary. In certain embodiments, complementary oligomeric compounds or regions are 100% complementary.

In advantageous embodiments, there is full or 100% complementarity between antisense and corresponding sense portions. In those instances where a sense portion is shorter than the corresponding antisense portion, such full complementarity is understood as referring to a number of base pairings which is identical to the number of nucleotides in said sense portion.

As regards complementarity of an antisense portion to a cognate mRNA, complementarity 5 is advantageously either full complementarity or there is one mismatch, said mismatch advantageously being located at the 5′ end of said antisense portion.

As used herein, “self-complementarity” in reference to oligomeric compounds means a compound that may fold back on itself, creating a duplex as a result of nucleobase hybridization of internal complementary strand regions. Depending on how close together and/or how long the strand regions are, then the compound may form hairpin loops, junctions, bulges or internal loops.

As used herein, “mismatch” means a nucleobase of an oligomeric compound that is not capable of pairing with a nucleobase at a corresponding position of a target sequence, or at a corresponding position of the oligomeric compound itself when the oligomeric compound hybridizes as a result of self-complementarity, when the oligomeric compound and the target sequence and/or self-complementary regions of the oligomeric compound, are aligned.

As used herein, “hybridization” means the pairing of complementary oligomeric compounds (e.g., an oligomeric compound and its target sequence). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site.

As used herein, “fully complementary” in reference to an oligomeric compound or region thereof means that each nucleobase of the oligomeric compound or region thereof is capable of pairing with a nucleobase of a complementary nucleic acid target sequence or a self-complementary region of the oligomeric compound. Thus, a fully complementary oligomeric compound or region thereof comprises no mismatches or unhybridized nucleobases with respect to its target sequence or a self-complementary region of the oligomeric compound.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression.

As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified naturally occurring RNA nucleoside are “differently modified,” even though the naturally occurring nucleoside is unmodified. Likewise, DNA and RNA oligonucleotides are “differently modified,” even though both are naturally occurring unmodified nucleosides. Nucleosides that are the same but for comprising different nucleobases are not differently modified. For example, a nucleoside comprising a 2′-0Me modified sugar moiety and an unmodified adenine nucleobase and a nucleoside comprising a 2′-OMe modified sugar moiety and an unmodified thymine nucleobase are not differently modified.

As used herein, “the same type of modifications” refers to modifications that are the same as one another, including absence of modifications. Thus, for example, two unmodified RNA nucleosides have “the same type of modification,” even though the RNA nucleosides are unmodified. Such nucleosides having the same type modification may comprise different nucleobases.

As used herein, “region” or “regions”, or “portion” or “portions”, mean a plurality of linked nucleosides that have a function or character as defined herein, in particular with reference to the claims and definitions as provided herein. Typically, such regions or portions comprise at least 10, at least 11, at least 12 or at least 13 linked nucleosides. For example, such regions can comprise 13 to 20 linked nucleosides, such as 13 to 16 or 18 to 20 linked nucleosides. Typically an antisense region as defined herein consists essentially of 18 to 20 nucleosides and a sense region as defined herein consists essentially of 13 to 16 linked nucleosides. Of note, first and second portions in accordance with the first aspect of the disclosed embodiments are antisense portions, and third and fourth portions are sense portions.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein, “substituent” and “substituent group,” means an atom or group that replaces the atom or group of a named parent compound. For example, a substituent of a modified nucleoside is any atom or group that differs from the atom or group found in a naturally occurring nucleoside (e.g., a modified 2′-substituent is any atom or group at the 2′-position of a nucleoside other than H or OH). Substituent groups can be protected or unprotected. In certain embodiments, compounds of the present disclosure have substituents at one or at more than one position of the parent compound. Substituents may also be further substituted with other substituent groups and may be attached directly or via a linking group such as oxygen or an alkyl or hydrocarbyl group to a parent compound.

Such substituents can be present as the modification on the sugar moiety, in particular a substituent present at the 2′-position of the sugar moiety. Unless otherwise indicated, groups amenable for use as substituents include without limitation, one or more of halo, hydroxyl, alkyl, alkenyl, alkynyl, acyl, carboxyl, alkoxy, alkoxyalkylene and amino substituents. Certain substituents as described herein can represent modifications directly attached to a ring of a sugar moiety (such as a halo, such as fluoro, directly attached to a sugar ring), or a modification indirectly linked to a ring of a sugar moiety by way of an oxygen linking atom that itself is directly linked to the sugar moiety (such as an alkoxyalkylene, such as methoxyethylene, linked to an oxygen atom, overall providing an MOE substituent as described herein attached to the 2′-position of the sugar moiety).

As used herein, “alkyl,” as used herein, means a saturated straight or branched monovalent C1-6 hydrocarbon radical, with methyl being a most advantageous alkyl as a substituent at the 2′-position of the sugar moiety. The alkyl group typically attaches to an oxygen linking atom at the 2′position of the sugar, therefore, overall providing a —Oalkyl substituent, such as an —OCH3 substituent, on a sugar moiety of an oligomeric compound according to the disclosed embodiments. This will be well understood be a person skilled in the art.

As used herein, “alkylene” means a saturated straight or branched divalent hydrocarbon radical of the general formula —CnH2n- where n is 1-6. Methylene or ethylene are advantageous alkylenes.

As used herein, “alkenyl” means a straight or branched unsaturated monovalent C2-6 hydrocarbon radical, with ethenyl or propenyl being most advantageous alkenyls as a substituent at the 2′-position of the sugar moiety. As will be well understood in the art, the degree of unsaturation that is present in an alkenyl radical is the presence of at least one carbon to carbon double bond. The alkenyl group typically attaches to an oxygen linking atom at the 2′-position of the sugar, therefore, overall providing a —O-alkenyl substituent, such as an —OCH₂CH═CH₂ substituent, on a sugar moiety of an oligomeric compound according to the disclosed embodiments. This will be well understood be a person skilled in the art.

As used herein, “alkynyl” means a straight or branched unsaturated C2-6 hydrocarbon radical, with ethynyl being a most advantageous alkynyl as a substituent at the 2′-position of the sugar moiety. As will be well understood in the art, the degree of unsaturation that is present in an alkynyl radical is the presence of at least one carbon to carbon triple bond. The alkynyl group typically attaches to an oxygen linking atom at the 2′-position of the sugar, therefore, overall providing a —Oalkynyl substituent on a sugar moiety of an oligomeric compound according to the disclosed embodiments. This will be well understood be a person skilled in the art.

As used herein, “carboxyl” is a radical having a general formula —CO₂H.

As used herein, “acyl” means a radical formed by removal of a hydroxyl group from a carboxyl radical as defined herein and has the general Formula —C(O)—X where X is typically C1-6 alkyl.

As used herein, “alkoxy” means a radical formed between an alkyl group, such as a C1-6 alkyl group, and an oxygen atom wherein the oxygen atom is used to attach the alkoxy group either to a parent molecule (such as at the 2′-position of a sugar moiety), or to another group such as an alkylene group as defined herein. Examples of alkoxy groups include without limitation, methoxy, ethoxy, propoxy, isopropoxy, n-butoxy, sec-butoxy and tert-butoxy. Alkoxy groups as used herein may optionally include further substituent groups.

As used herein, alkoxyalkylene means an alkoxy group as defined herein that is attached to an alkylene group also as defined herein, and wherein the oxygen atom of the alkoxy group attaches to the alkylene group and the alkylene attaches to a parent molecule. The alkylene group typically attaches to an oxygen linking atom at the 2′-position of the sugar, therefore, overall providing a —Oalkylenealkoxy substituent, such as an —OCH2CH₂OCH3 substituent, on a sugar moiety of an oligomeric compound according to the disclosed embodimentsn. This will be well understood by a person skilled in the art and is generally referred to as an MOE substituent as defined herein and as known in the art.

As used herein, “amino” includes primary, secondary and tertiary amino groups.

As used herein, “halo” and “halogen,” mean an atom selected from fluorine, chlorine, bromine and iodine.

As used herein, the term “mxRNA” is in particular understood as defined in WO 2020/044186 A2, which is incorporated by reference herein in its entirety. In particular, an mxRNA is a hairpin-shaped RNA molecule consisting of an antisense portion (also referred to as the guide strand) and a sense portion (also referred to the passenger strand). The mxRNA comprises duplex region and a hairpin loop, wherein said mxRNA has an approximate length of about 34 nucleotides. Said duplex region comprises a region in which parts of the antisense portion and substantially the entire sense portion, typically 14 or 15 nucleotides of each strand, are base-paired. Said hairpin loop connects both regions, i.e., antisense region and sense region, of that duplex via e.g. a phosphate or a phosphorothioate linker, i.e., covalently, while the antisense portion typically has a length of about 18 to 20 nucleotides and, therefore, forms the antisense duplex region and the loop. The loop, of which the antisense portion is part, furthermore connects the sense, forming the second strand of the loop, and the antisense portion.

The term “angiotensinogen” or abbreviated “AGT”, also known as SERPINA 8 or ANHU, is used in its common sense and denotes a protein produced in the liver which is a component of the renin-angiotensin-aldosterone-system (RAAS), and which is converted to angiotensin I by renin when released in circulation. The identifier in the RefSeq database of the human AGT mRNA is NM_000029. Angiotensinogen is expressed and produced in the liver by the angiontensin gene or “AGT gene”.

The term “apolipoprotein C3” or abbreviated “APOC3” has its art-established meaning. The identifier in the RefSeq database of the human APOC3 mRNA is NM_000040. It is secreted by the liver and the small intestine. It can be found on triglyceride-rich lipoproteins including very low density lipoproteins (VLDL) and chylomicrons. It is involved in the negative regulation of lipid catabolismespecially triglyceride catabolismand of the clearance of VLDL, LDL and HDL lipoproteins. A molecular function of APOC3 is the inhibition of lipoprotein lipase and of hepatic lipase.

As used herein, the term “muRNA” or “multi RNA” includes nucleic acid constructs comprising more than one, typically two, RNA sequences, i.e., first and second nucleic acid portions, targeting different regions of AGT mRNA; or one region of AGT mRNA and an mRNA region of another target molecule, here advantageous APOC3. The targeting RNA sequences are also referred to as “antisense” or “guide” strands, while the respective passenger strands, i.e., third and fourth nucleic acid portions being complementary to the first and second portion, respectively, are also included in the nucleic acid construct. In particular, such muRNA are designed such that subsequent to in vivo administration, they are disassembled and said first and second nucleic acid portions are released. A particular example for such muRNA is shown below, where (1) is the first nucleic acid portion, (2) is the third nucleic acid portion being complementary to (1), (3) is the second nucleic acid portion being complementary to the fourth nucleic acid portion, while (5) is a labile linker while (6) is a ligand, which will both be explained below.

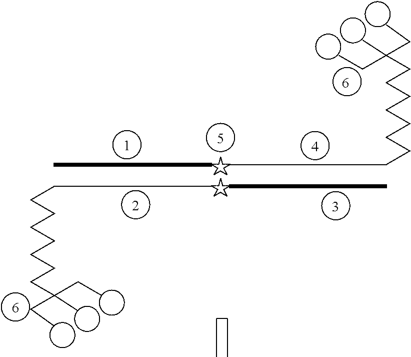

Further miniaturization by shortening the sense regions leads to bulge in the central part of the molecule where the 3′-terminal regions of the two antisense regions face each other:

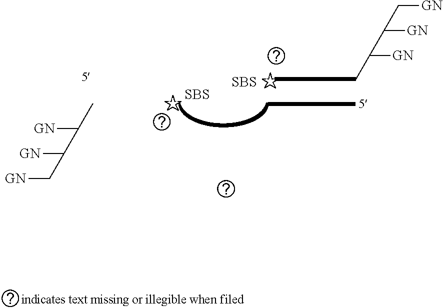

In the diagram above, “GN” designates a GalNAc moiety, and “SBS” designates the fragile site which may be implemented as a nucleoside with a non-modified sugar.

It will also be understood that oligomeric compounds as described herein may have one or more non-hybridizing nucleosides at one or both ends of one or both strands (overhangs) and/or one or more internal non-hybridizing nucleosides (mismatches) provided there is sufficient complementarity to maintain hybridization under physiologically relevant conditions. Alternatively, oligomeric compounds as described herein may be blunt ended at least one end. Particularly advantageous is that both ends are blunt.

The term “comprising” is used herein to mean including the method steps or elements identified, but that such steps or elements do not comprise an exclusive list and as such, there may be present additional steps or elements.

Further, to the extent that the term “includes” is used in either the detailed description or the claims, such term is intended to be inclusive in a manner similar to the term “comprising” as “comprising” is interpreted when employed as a transitional word in a claim.

Non-Limiting Aspects and Embodiments muRNA Nucleic Acid Constructs

According to a first aspect, the disclosed embodiments are directed to a nucleic acid construct comprising at least:
(a) a first nucleic acid portion that is at least partially complementary to at least a first portion of an RNA, which is transcribed from an APOC3 gene;
(b) a second nucleic acid portion that is at least partially complementary to at least a second portion of an RNA, which is transcribed from an AGT gene;
(c) a third nucleic acid portion that is at least partially complementary to said first nucleic acid portion of (a), so as to form a first nucleic acid duplex region therewith;
(d) a fourth nucleic acid portion that is at least partially complementary to said second nucleic acid portion of (b), so as to form a second nucleic acid duplex region therewith.

Said construct may be designed such that subsequent to in vivo administration said construct disassembles to yield at least first and second discrete nucleic acid targeting molecules that respectively target said RNA portions transcribed from said target genes of (a) and (b); whereby (i) said first nucleic acid targeting molecule is capable of modulating expression of said target gene of (a), and comprises, or is derived from, at least said first nucleic acid portion of (a), and (ii) said second nucleic acid targeting molecule is capable of modulating expression of said target gene of (b), and comprises, or is derived from, said second nucleic acid portion of (b).

Said construct may be designed to disassemble such that said first and second discrete nucleic acid targeting molecules are respectively processed by independent RNAi-induced silencing complexes.

### Sequence Features, Labile Functionality, and Structural Features of the RNA Molecules

The construct according to the first aspect and its aforementioned embodiments may at least comprise one labile functionality such that subsequent to in vivo administration said construct is cleaved so as to yield said at least first and second discrete nucleic acid targeting molecules.

Said labile functionality may comprise one or more unmodified nucleotides. In particular said one or more unmodified nucleotides of said labile functionality represent one or more cleavage positions within said construct whereby subsequent to in vivo administration said construct is cleaved at said one or more cleavage positions so as to yield said at least first and second discrete nucleic acid targeting molecules. Especially, said cleavage positions may be respectively located within the construct so that subsequent to cleavage said first discrete nucleic acid targeting molecule comprises, or is derived from, said first nucleic acid duplex region, and said second discrete nucleic acid targeting molecule comprises, or is derived from, said second nucleic acid duplex region. Advantageously, said first discrete nucleic acid targeting molecule comprises or consists of said first nucleic acid portion of (a) and said third nucleic acid portion of (c), and/or said second discrete nucleic acid targeting molecule comprises or consists of said second nucleic acid portion of (b) and said fourth nucleic acid portion of (d).

In certain embodiments
(a) said first nucleic acid portion has a nucleobase sequence selected from SEQ ID NOs: 1 to 30;
(b) said second nucleic acid portion has a nucleobase sequence selected from SEQ ID NOs: 61 to 90;
(c) said third nucleic acid portion has a nucleobase sequence selected from SEQ ID NOs: 31 to 60; and/or
(d) said fourth nucleic acid portion has a nucleobase sequence selected from SEQ ID NOs: 91 to 120.

These sequences have been found to perform outstandingly when used in the context of molecules which trigger RNA interference and are directed to a single target; see, for example, PCT/US2022/34965 (for APOC3 targeting molecules) and U.S. 63/407,353 (for AGT targeting molecules), respectively. These two patent document are incorporated by reference herewith. Since the present inventors surprisingly found in several instances that outstanding performance in single-targeting molecules (such as mxRNAs) may be transferred to double-targeting molecules (such as muRNAs), any further sequences, in particular antisense sequences as disclosed in the above-mentioned patent documents may serve as a basis for designing muRNAs of the disclosed embodiments.

As explained above, said third and fourth nucleobase sequences, to the extent they have a length of 14 nucleobases, are advantageous extended by one nucleotide (at the 5′ end of said third and fourth sequences, following the guidance given above). The compounds which have been shown to be active as single-targeting molecules in the APOC3 inhibition have been demonstrated to be surprisingly highly active when used within a degradable nucleic acid construct according to the disclosed embodiments.

In certain such embodiments, said first nucleic acid portion of (a) may be directly or indirectly linked to said fourth nucleic acid portion of (d) as a primary structure.

In certain embodiments, said second nucleic acid portion of (b) may be directly or indirectly linked to said third nucleic acid portion of (c) as a primary structure.

In certain embodiments, said first nucleic acid portion has the nucleobase sequence of SEQ ID NO: 3.

In certain embodiments, said second nucleic acid portion has a nucleobase sequence selected from SEQ ID NOs: 61, 66, 67, 73, 68 and 76.

In certain embodiments, said third nucleic acid portion has the nucleobase sequence of SEQ ID NO: 33.

In certain embodiments, said fourth nucleic acid portion has a nucleobase sequence selected from SEQ ID NOs: 91, 96, 97, 103, 98 and 106.

As stated above, it is advantageous that sense sequences of third and fourth portions have a length of 15 nucleotides. To the extent the above specified entries of the sequence listing have a length of 14 nucleotides, a further nucleotide is to be added at the 5′ end of the respective portion, said further nucleotide being complementary to nucleotide at position 15 of the corresponding antisense portion (which is the first portion in case of the third portion, and the second portion in case of the fourth portion).

In certain embodiments, said construct may further comprise 1 to 8 additional nucleic acid portions that are respectively at least partially complementary to an additional 1 to 8 portions of RNA transcribed from one or more target genes, which target genes may be the same or different to each other, and/or the same or different to the target genes defined in (a) and/or
(b), and wherein each of the 1 to 8 additional nucleic acid portions respectively form additional duplex regions with respective passenger nucleic acid portions that are respectively at least partially complementary therewith. In particular, said second nucleic acid portion of (b), and said 1 to 8 additional nucleic acid portions, may be directly or indirectly linked to selected passenger nucleic acid portions as respective primary structures.

In certain embodiments said direct or indirect linking may represent either (i) an internucleotide bond, (ii) an internucleotide nick, or (iii) a nucleic acid linker portion of 1, 2, 3, 4, 5, 6, 7, 8, 9, or 10 nucleotides, said nucleic acid linker advantageously being single stranded.

Advantageously, said linking may be direct, thereby giving rise to (a) contiguous strand(s). In certain embodiments, there may exist some complementarity between the first nucleic acid portion of (a) and the second nucleic acid portion of (b), or the third nucleic acid portion of
(c) and the fourth nucleic acid portion of (d). Advantageously, said complementarity:
(i) may be 1, 2, 3, 4, 5, 6, 7, 8, 9, or 10, advantageously 2, 3, 4 or 5 base pairs; and/or
(ii) may be between the first nucleic acid portion of (a) and the second nucleic acid portion of (b).

In certain embodiments, said internucleotide bond may involve at least one of said one or more unmodified nucleotides, wherein advantageously cleavage may occur at the 3′ position of (at least one of) said unmodified nucleotide(s).

In certain embodiments, said first nucleic acid portion of (a), and/or said second nucleic acid portion of (b), and/or said third nucleic acid portion of (c), and/or said fourth nucleic acid portion of (d), may be respectively 7 to 25 nucleotides in length. Optionally, said first nucleic acid portion of (a) and/or said second nucleic acid portion of (b) may have a length of 18 to 21, more advantageously 18 to 20, and yet more advantageously 19 nucleotides. In advantageous embodiments, said first nucleic acid portion of (a) and said second nucleic acid portion of (b) have a length of 19 nucleotides. It may be further advantageous that said third nucleic acid portion of (c), and/or said fourth nucleic acid portion of (d) have a length of 11 to 20, more advantageously 13 to 16, and yet more advantageously 14 or 15, most advantageously 15 nucleotides.

In certain embodiments, said first nucleic portion of (a) and said second nucleic acid portion of (b) may have a length of 19 nucleotides and said third nucleic acid portion of (c) as well as said fourth nucleic acid portion of (b) may have a length of 15 nucleotides.

In certain embodiments, said unmodified nucleotide(s) is/are at any of position 18 to 25, more advantageously at any of positions 18 to 21, and/or the 3′ terminal position of said first nucleic acid portion of (a) and/or of said second nucleic acid portion of (b).

In certain embodiments, said unmodified nucleotide is at position 19.

In certain embodiments, said first nucleic portion of (a) and said second nucleic acid portion of (b) may have a length of 19 nucleotides and said third nucleic acid portion of (c) as well as said fourth nucleic acid portion of (b) may have a length of 15 nucleotides and said unmodified nucleoside is at position 19 of said first nucleic acid portion of (a) and said second nucleic acid portion of (b).

In certain embodiments, said nucleic acid linker portion may be 1 to 8 nucleotides in length, advantageously 2 to 7 or 3 to 6 nucleotides in length, more advantageously about 4 or 5 and most advantageously 4 nucleotides in length.

In certain embodiments, one, more of all of the duplex regions independently may have a length of 10 to 19, more advantageously 13 to 19, and yet more advantageously 13, 14 or 15 base pairs, most advantageously 15 base pairs, wherein optionally there is one mismatch within said duplex region.

In certain embodiments, the nucleic acid construct may be blunt ended.

In certain embodiments, the first nucleic acid portion of (a); and/or the second nucleic acid portion of (b); and/or the third nucleic acid portion of (c); and/or the fourth nucleic acid portion of (d); and/or to the extent present, the 1 to 8 additional nucleic acid portions as defined previously herein; and/or to the extent present, the passenger nucleic acid portions as defined previously herein; may have an overhang.

In certain embodiments, the target RNA may be an mRNA or another RNA molecule.

In certain embodiments, said construct comprises two strands, wherein the nucleobase sequences of first and second strand are SEQ ID NO: 130 and 131, SEQ ID NO: 132 and 133, SEQ ID NO: 134 and 135, SEQ ID NO: 136 and 137, SEQ ID NO: 138 and 139, SEQ ID NO: 140 and 141, SEQ ID NO: 142 and 143, SEQ ID NO: 144 and 145, or SEQ ID NO: 146 and 147, respectively.

Sequences referenced herein are shown in Tables 1 and 2 below. The sequence names in Table 1 denote whether the sequence shown is the sense(s) or antisense (as) strand. In particularly advantageous embodiments, taking into account information about modifications of the sugar-phosphate backbone, constructs of the disclosed embodiments are those shown in Table 2 below. Particularly advantageous among those are the constructs labelled Apo28-AGT27A and Apo28-AGT27. Performance of all constructs of Table 2 is shown in the Examples.

TABLE 1

| SEQ | | |
|---|---|---|
| ID No. | Name: | Sequence |
| 1 | APOC3 277 as | ttggataggc aggtggact |
| 2 | APOC3 337 as | tgcactgaga atactgtcc |
| 3 | APOC3 28 as | tcaacaagga gtacccggg |
| 4 | APOC3 343 as | taggagagca ctgagaata |
| 5 | APOC3 369 as | tcttgtccag ctttattgg |
| 6 | APOC3 366 as | ttccagcttt attgggagg |
| 7 | APOC3 274 as | tataggcagg tggacttgg |
| 8 | APOC3 367 as | tgtccagctt tattgggag |
| 9 | APOC3 336 as | tcactgagaa tactgtccc |
| 10 | APOC3 332 as | tgagaatact gtccctttt |
| 11 | APOC3 293 as | tcaaggagct cgcaggatg |
| 12 | APOC3 373 as | tgcttcttgt ccagcttta |
| 13 | APOC3 280 as | tggatggata ggcaggtgg |
| 14 | APOC3 221 as | tcagaactca gagaacitc |
| 15 | APOC3 334 as | tctgagaata ctgtccctt |
| 16 | APOC3 286 as | tctcgcagga tggataggc |
| 17 | APOC3 149 as | tcctgcacgc tgctcagtg |
| 18 | APOC3 193 as | tgctccagta gtctttcag |
| 19 | APOC3 328 as | tatactgtcc cttttaagc |
| 20 | APOC3 175 as | tggaactgaa gccatcggt |
| 21 | APOC3 262 as | ttctcaggca gccacggct |
| 22 | APOC3 254 as | tcacggctga agttggtct |
| 23 | APOC3 185 as | tagtctttca gggaactga |
| 24 | APOC3 328 as | tatactgtcc cttttaagc |
| 25 | APOC3 271 as | tggtattgag gtctcaggc |
| 26 | APOC3 137 as | ttcagtgcat ccttggcgg |
| 27 | APOC3 225 as | tatcccagaa ctcagagaa |
| 28 | APOC3 167 as | tagccatcgg tcacccagc |
| 29 | APOC3 297 as | tgacccaagg agctcgcag |
| 30 | APOC3 191 as | ttccagtagt ctttcaggg |
| 31 | APOC3 277 s | cacctgccta tccaa |
| 32 | APOC3 337 s | agtattctca gtgca |
| 33 | APOC3 28 s | ggtactcctt gttga |
| 34 | APOC3 343 s | ctcagtgctc tccta |
| 35 | APOC3 369 s | taaagctgga caaga |
| 36 | APOC3 366 s | ccaataaagc tggaa |
| 37 | APOC3 274 s | gtccacctgc ctata |
| 38 | APOC3 367 s | caataaagct ggaca |
| 39 | APOC3 336 s | cagtattctc agtga |
| 40 | APOC3 332 s | gggacagtat tctca |
| 41 | APOC3 293 s | ctgcgagctc cttga |
| 42 | APOC3 373 s | gctggacaag aagca |
| 43 | APOC3 280 s | ctgcctatcc atcca |
| 44 | APOC3 221 s | ttctctgagt tctga |
| 45 | APOC3 334 s | gacagtattc tcaga |
| 46 | APOC3 286 s | atccatcctg cgaga |
| 47 | APOC3 149 s | gagcagcgtg cagga |
| 48 | APOC3 193 s | aagactactg gagca |
| 49 | APOC3 328 s | aaaagggaca gtata |
| 50 | APOC3 175 s | atggcttcag ttcca |
| 51 | APOC3 262 s | gtggctgcct gagaa |
| 52 | APOC3 254 s | caacttcagc cgtga |
| 53 | APOC3 185 s | ttccctgaaa gacta |
| 54 | APOC3 328 s | aaaagggaca gtata |
| 55 | APOC3 271 s | gagacctcaa tacca |
| 56 | APOC3 137 s | caaggatgca ctgaa |
| 57 | APOC3 225 s | ctgagttctg ggata |
| 58 | APOC3 167 s | ggtgaccgat ggcta |
| 59 | APOC3 297 s | gagctccttg ggtca |
| 60 | APOC3 191 s | gaaagactac tggaa |
| 61 | AGT 27 as | ttagaagaaa aggtcggag |
| 62 | AGT 44 as | gttgagggag ttttgctgg |
| 63 | AGT 41 as | aaacactggt tcttgcctc |
| 64 | AGT 97 as | ccgcgctaaa cactggttc |
| 65 | AGT 90 as | ctggaaagtg agaccctcc |
| 66 | AGT 62 as | ttcacaaaca agctggtcg |
| 67 | AGT 52 as | ttttgttctc aacttgaaa |
| 68 | AGT 93 as | ggtactctca ttctggatg |
| 69 | AGT 49 as | actctgtggg ctctctctc |
| 70 | AGT 73 as | acaggtactc tcattctgg |
| 71 | AGT 18 as | caaacaagct ggtcggttg |
| 72 | AGT 37 as | gggctctctc tcatccgct |
| 73 | AGT 56 as | cagcaaacag gaatgggcg |
| 74 | AGT 100 as | cttgaaaagg gaacacttt |
| 75 | AGT 40 as | ctttaatttt aaaacccaa |
| 76 | AGT 75 as | atgaacctgt caatcttct |
| 77 | AGT 30 as | acaaacaagc tggtcggtt |
| 78 | AGT 42 as | caagctggtc ggttggaat |
| 79 | AGT 81 as | gtagactctg tgggctctc |
| 80 | AGT 17 as | tctcaacttg aaaagggaa |
| 81 | AGT 34 as | aaaatgctgt tcagcacct |
| 82 | AGT 53 as | gagggagttt tgctggaaa |
| 83 | AGT 29 as | agagaggcca gggtgccaa |
| 84 | AGT 26 as | ccttctgctg tagtaccca |
| 85 | AGT 74 as | cggaagccca agaagttgg |
| 86 | AGT 94 as | ttccatcctg tcacagcct |
| 87 | AGT 14 as | aaacaagctg gtcggttgg |
| 88 | AGT 3 as | tgaggtgctg ttgtccacc |
| 89 | AGT 7 as | cactgaggtg ctgttgtcc |
| 90 | AGT 2 as | ggtgctgttg tccacccag |
| 91 | AGT 27 s | accttttctt ctaa |
| 92 | AGT 44 s | aaaactccct caac |
| 93 | AGT 41 s | aagaaccagt gttt |
| 94 | AGT 97 s | agtgtttagc gcgg |
| 95 | AGT 90 s | gtctcacttt ccag |
| 96 | AGT 62 s | agcttgtttg tgaa |
| 97 | AGT 52 s | agttgagaac aaaa |
| 98 | AGT 93 s | acaatgagag tacc |
| 99 | AGT 49 s | agagcccaca gagt |
| 100 | AGT 73 s | atgagagtac ctgt |
| 101 | AGT 18 s | gaccagcttg tttg |
| 102 | AGT 37 s | atgagagaga gccc |
| 103 | AGT 56 s | attcctgttt gctg |
| 104 | AGT 100 s | gttccctttt caag |
| 105 | AGT 40 s | ttttaaaatt aaag |
| 106 | AGT 75 s | attgacaggt tcat |
| 107 | AGT 30 s | accagcttgt ttgt |
| 108 | AGT 42 s | aaccgaccag cttg |
| 109 | AGT 81 s | cccacagagt ctac |
| 110 | AGT 17 s | ttttcaagtt gaga |
| 111 | AGT 34 s | ctgaacagca tttt |
| 112 | AGT 53 s | agcaaaactc cctc |
| 113 | AGT 29 s | accctggcct ctct |
| 114 | AGT 26 s | actacagcag aagg |
| 115 | AGT 74 s | ttcttgggct tccg |
| 116 | AGT 94 s | gtgacaggat ggaa |
| 117 | AGT 14 s | cgaccagctt gitt |
| 118 | AGT 3 s | acaacagcac ctca |
| 119 | AGT 7 s | acagcacctc agtg |
| 120 | AGT 2 s | tggacaacag cacc |
| 121 | AGT 27 mx | ttagaagaaa aggtgggaga ccttttcttc taa |
| 122 | AGT 62 mx | ttcacaaaca agctggtcga gcttgtttct gaa |
| 123 | AGT 52 mx | ttttgttctc aacttgaaaa gttgagaaca aaa |
| 124 | AGT 56 mx | tagcaaacag gaatgggcga ttcctgtitg cta |
| 125 | AGT 93 mx | tgtactctca ttgtggatga caatgagagt aca |
| 126 | AGT 75 mx | ttgaacctgt caatcttcta ttgacaggtt caa |
| 127 | AGT 27A mx | atagaagaaa aggtgggaga ccttttcttc tat |
| 128 | AGT 62A mx | atcacaaaca agctggtcga gcttgtttgt gat |
| 129 | AGT 52A mx | atttgttctc aacttgaaaa gttgagaaca aat |
| 130 | Apo28-AGT27 Apo 28 mu as | tcaacaagga gtacccgggc accttttctt ctaa |
| 131 | Apo28-AGT27 AGT 27 mu as | ttagaagaaa aggtgggagg gtactccttg ttga |
| 132 | Apo28-AGT62 Apo28 mu as | tcaacaagga gtacccgggc agcttgtttg tgaa |
| 133 | Apo28-AGT62 AGT 62 mu as | ttcacaaaca agctggtcgg gtactccttg ttca |
| 134 | Apo28-AGT52 Apo 28 mu as | tcaacaagga gtacccggga agttgagaac aaaa |
| 135 | Apo28-AGT52 AGT 52 mu as | ttttgttctc aacttgaaag gtactccttg ttga |
| 136 | Apo28-AGT75 Apo 28 mu as | tcaacaagga gtacccgggg attgacaggt tcaa |
| 137 | Apo28-AGT75 AGT 75 mu as | ttgaacctgt caatcttctg gtactccttg ttga |
| 138 | Apo28-AGT56 Apo 28 mu as | tcaacaagga gtacccgggc attcctgttt gcta |
| 139 | Apo28-AGT56 AGT 56 mu as | tagcaaacag gaatgggcgg gtactccttg ttga |
| 140 | Apo28-AGT93 Apo 28 mu as | tcaacaagga gtacccgggc acaatgagag taca |
| 141 | Apo28-AGT93 AGT 93 mu as | tgtactctca ttgtggatgg gtactccttg ttga |
| 142 | Apo28-AGT27A Apo 28 | tcaacaagga gtacccgggc accttttctt ctat |
| | mu as | |
| 143 | Apo28-AGT27A AGT 27A | atagaagaaa aggtgggagg gtactccttg ttga |
| | mu as | |
| 144 | Apo28-AGT62A Apo 28 | tcaacaagga gtacccgggc agcttgtttg tgat |
| | mu as | |
| 145 | Apo28-AGT62A AGT 62A | atcacaaaca agctggtcgg gtactcctty ttga |
| | mu as | |
| 146 | Apo28-AGT52A Apo 28 | tcaacaagga gtacccggga agttgagaac aaat |
| | mu as | |
| 147 | Apo28-AGT52A AGT52A | atttgttctc aacttgaaag gtactccttg ttga |
| | mu as | |

TABLE 2
Advantageous muRNAs of the disclosed embodiments. Each construct comprises
two strands as shown below.

| Construct/ | |
|---|---|
| SEQ ID No | Modified sequences, 5′ to 3′ |
| | Name: Apo28-AGT27 |
| 148 | [mU][Ps][fU][Ps][mA][fG][mA][fA][mG][fA][mA][fA][mA][fG] |
| | [mG][fU][mG][Ps][fG][Ps][mG][Ps][fA][Ps][rG][fG][Ps][mG][Ps] |
| | [fU][mA][fC][mU][fC][mC][fU][mU][fG][mU][fU][Ps][mG][Ps] |
| | [fA][Ps][3XGalNAc] |
| 149 | [mU][Ps][fC][Ps][mA][fA][mC][fA][mA][fG][mG][fA][mG][fU] |
| | [mA][fC][mC][Ps][fC][Ps][mG][Ps][fG][Ps][rG][fC][Ps][mA][Ps] |
| | [fC][mC][fU][mU][fU][mU][fC][mU][fU][mC][fU][Ps][mA][Ps] |
| | [fA][Ps][3XGalNAc] |
| | Name: Apo28-AGT62 |
| 150 | [mU][Ps][fU][Ps][mC][fA][mC][fA][mA][fA][mC][fA][mA][fG] |
| | [mC][fU][mG][Ps][fG][Ps][mU][Ps][fC][Ps][fG][fG][Ps][mG][Ps] |
| | [fU][mA][fC][mU][fC][mC][fU][mU][fG][mU][fU][Ps][mG][Ps] |
| | [fA][Ps][3XGalNAc] |
| 151 | [mU][Ps][fC][Ps][mA][fA][mC][fA][mA][fG][mG][fA][mG][fU] |
| | [mA][fC][mC][Ps][fC][Ps][mG][Ps][fG][Ps][fG][fC][Ps][mA][Ps] |
| | [fG][mC][fU][mU][fG][mU][fU][mU][fG][mU][fG][Ps][mA][Ps] |
| | [fA][Ps][3XGalNAc] |
| | Name: Apo28-AGT52 |
| 152 | [mU][Ps][fU][Ps][mU][fU][mG][fU][mU][fC][mU][fC][mA][fA] |
| | [mC][fU][mU][Ps][fG][Ps][mA][Ps][fA][Ps][rA][fG][Ps][mG][Ps] |
| | [fA][mA][fC][mU][fC][mC][fU][mU][fG][mU][fU][Ps][mG][Ps] |
| | [fA][Ps][3XGalNAc] |
| 153 | [mU][Ps][fC][Ps][mA][fA][mC][fA][mA][fG][mG][fA][mG][fU] |
| | [mA][fC][mC][Ps][fC][Ps][mG][Ps][fG][Ps][[G][fA][Ps][mA][Ps] |
| | [fG][mU][fU][mG][fA][mG][fA][mA][fC][mA][fA][Ps][mA][Ps] |
| | [fA][Ps][3XGalNAc] |
| | Name: Apo28-AGT75 |
| 154 | [mU][Ps][fU][Ps][mG][fA][mA][fC][mC][fU][mG][fU][mC][fA] |
| | [mA][fU][mC][Ps][fU][Ps][mU][Ps][fC][Ps][rU][fG][Ps][mG][Ps] |
| | [fU][mA][fC][mU][fC][mC][fU][mU][fG][mU][fU][Ps][mG][Ps] |
| | [fA][Ps][3XGalNAc] |
| 155 | [mU][Ps][fC][Ps][mA][fA][mC][fA][mA][fG][mG][fA][mG][fU] |
| | [mA][fC][mC][Ps][fC][Ps][mG][Ps][fG][Ps][rG][fG][Ps][mA][Ps] |
| | [fU][mU][fG][mA][fC][mA][fG][mG][fU][mU][fC][Ps][mA][Ps] |
| | [fA][Ps][3XGalNAc] |
| | Apo28-AGT56 |
| 156 | [mU][Ps][fA][Ps][mG][fC][mA][fA][mA][fC][mA][fG][mG][fA] |
| | [mA][fU][mG][Ps][fG][Ps][mG][Ps][fC][Ps][fG][fG][Ps][mG][Ps] |
| | [fU][mA][fC][mU][fC][mC][fU][mU][fG][mU][fU][Ps][mG][Ps] |
| | [fA][Ps][3XGalNAc] |
| 157 | [mU][Ps][fC][Ps][mA][fA][mC][fA][mA][fG][mG][fA][mG][fU] |
| | [mA][fC][mC][Ps][fC][Ps][mG][Ps][fG][Ps][fG][fC][Ps][mA][Ps] |
| | [fU][mU][fC][mC][fU][mG][fU][mU][fU][mG][fC][Ps][mU][Ps] |
| | [fA][Ps][3XGalNAc] |
| | Apo28-AGT93 |
| 158 | [mU][Ps][fG][Ps][mU][fA][mC][fU][mC][fU][mC][fA][mU][fU] |
| | [mG][fU][mG][Ps][fG][Ps][mA][Ps][fU][Ps][fG][fG][Ps][mG][Ps] |
| | [fU][mA][fC][mU][fC][mC][fU][mU][fG][mU][fU][Ps][mG][Ps] |
| | [fA][Ps][3XGalNAc] |
| 159 | [mU][Ps][fC][Ps][mA][fA][mC][fA][mA][fG][mG][fA][mG][fU] |
| | [mA][fC][mC][Ps][fC][Ps][mG][Ps][fG][Ps][fG][fC][Ps][mA][Ps] |
| | [fC][mA][fA][mU][fG][mA][fG][mA][fG][mU][fA][Ps][mC][Ps] |
| | [fA][Ps][3XGalNAc] |
| | Apo28-AGT27A |
| 160 | [mA][Ps][fU][Ps][mA][fG][mA][fA][mG][fA][mA][fA][mA][fG] |
| | [mG][fU][mG][Ps][fG][Ps][mG][Ps][fA][Ps][fG][fG][Ps][mG][Ps] |
| | [fU][mA][fC][mU][fC][mC][fU][mU][fG][mU][fU][Ps][mG][Ps] |
| | [fA][Ps][3XGalNAc] |
| 161 | [mU][Ps][fC][Ps][mA][fA][mC][fA][mA][fG][mG][fA][mG][fU] |
| | [mA][fC][mC][Ps][fC][Ps][mG][Ps][fG][Ps][rG][fC][Ps][mA][Ps] |
| | [fC][mC][fU][mU][fU][mU][fC][mU][fU][mC][fU][Ps][mA][Ps] |
| | [fU][Ps][3XGalNAc] |
| | Apo28-AGT62A |
| 162 | [mA][Ps][fU][Ps][mC][fA][mC][fA][mA][fA][mC][fA][mA][fG] |
| | [mC][fU][mG][Ps][fG][Ps][mU][Ps][fC][Ps][rG][fG][Ps][mG][Ps] |
| | [fU][mA][fC][mU][fC][mC][fU][mU][fG][mU][fU][Ps][mG][Ps] |
| | [fA][Ps][3XGalNAc] |
| 163 | [mU][Ps][fC][Ps][mA][fA][mC][fA][mA][fG][mG][fA][mG][fU] |
| | [mA][fC][mC][Ps][fC][Ps][mG][Ps][fG][Ps][rG][fC][Ps][mA][Ps] |
| | [fG][mC][fU][mU][fG][mU][fU][mU][fG][mU][fG][Ps][mA][Ps] |
| | [fU][Ps][3XGalNAc] |
| | Name: Apo28-AGT62A |
| 164 | [mA][Ps][fU][Ps][mU][fU][mG][fU][mU][fC][mU][fC][mA][fA] |
| | [mC][fU][mU][Ps][fG][Ps][mA][Ps][fA][Ps][fA][fG][Ps][mG][Ps] |
| | [fU][mA][fC][mU][fC][mC][fU][mU][fG][mU][fU][Ps][mG][Ps] |
| | [fA][Ps][3XGalNAc] |
| 165 | [mU][Ps][fC][Ps][mA][fA][mC][fA][mA][fG][mG][fA][mG][fU] |
| | [mA][fC][mC][Ps][fC][Ps][mG][Ps][fG][Ps][rG][fA][Ps][mA][Ps] |
| | [fG][mU][fU][mG][fA][mG][fA][mA][fC][mA][fA][Ps][mA][Ps] |
| | [fU][Ps][3XGalNAc] |

[mN], N being any nucleoside, designates 2′-0Me; [IN], N being any nucleoside, designates: 2′-F; [rN], N being any nucleoside, designates: 2′-OH; [Ps] designates a phosphorothioate connecting two adjacent nucleosides; and [3XGalNAc] designates the following ligand, wherein the strand to which said ligand is bound is shown in square brackets:

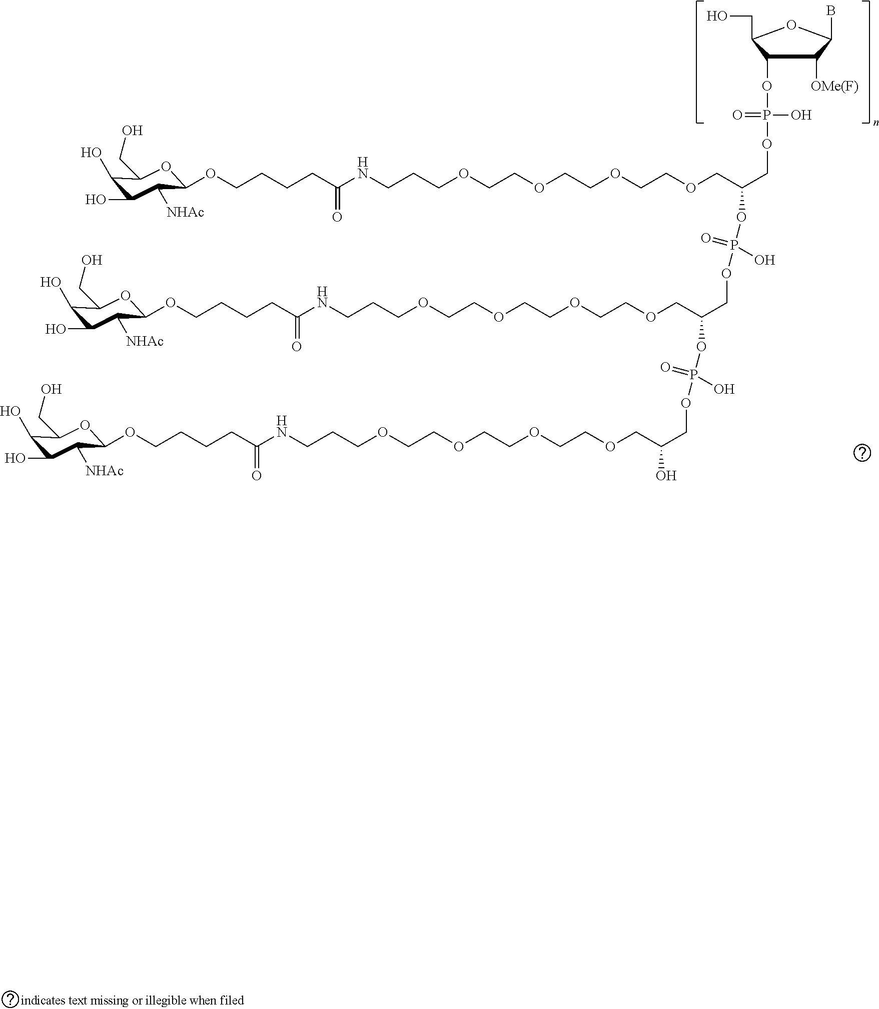

### Ligands

The nucleic acid construct according to the second aspect and the aforementioned embodiments may further comprise one or more ligands.

In certain embodiments, said first nucleic acid portion of (a), and/or said second nucleic acid portion of (b), and/or said third nucleic acid portion of (c), and/or said fourth nucleic acid portion of (d), and/or, to the extent present, said 1 to 8 additional nucleic acid portions as defined previously herein, and/or said passenger nucleic acid portions as defined previously herein, respectively may have a 5′ to 3′ directionality thereby defining 5′ and 3′ regions thereof.

In certain embodiments, one or more ligands are conjugated at the 31region, advantageously the 3′ end, of any of (i) said third nucleic acid portion of (c), and/or (ii) said fourth nucleic acid portion of (d), and/or, to the extent present, said (iii) passenger nucleic acid portions as defined previously herein.

In certain embodiments, one or more ligands may be conjugated at one or more regions intermediate of the 5′ and 3′ regions of any of said nucleic acid portions, advantageously of said third nucleic acid portion of (c), and/or said fourth nucleic acid portion of (d), and/or said passenger nucleic acid portions as defined previously herein.

In certain embodiments, one or more ligands may be conjugated at the 5′ region, advantageously the 5′ end, of any of said nucleic acid portions.

In certain embodiments, said one or more ligands may be any cell directing moiety, such as lipids, carbohydrates, aptamers, vitamins and/or peptides that bind cellular membrane or a specific target on cellular surface. In an advantageous embodiment, said one or more carbohydrates can be a monosaccharide, disaccharide, trisaccharide, tetrasaccharides, oligosaccharide or polysaccharide. In a more advantageous embodiment, said one or more carbohydrates may comprise one or more hexose moieties. Especially, said one or more hexose moieties may be one or more galactose moieties, one or more lactose moieties, one or more N-Acetyl-Galactosamine moieties, and/or one or more mannose moieties. Said hexose moiety may be comprise two or three N-Acetyl-Galactosamine moieties. In particular, said hexose moiety may comprise three N-Acetyl-Galactosamine moieties.

In certain embodiments, said one or more ligands may be attached in a linear configuration, or in a branched configuration. Advantageously, wherein said one or more ligands may be attached as a biantennary or triantennary configuration, or as a configuration based on single ligands at different positions.

Advantageously, said ligand may have the following structure:

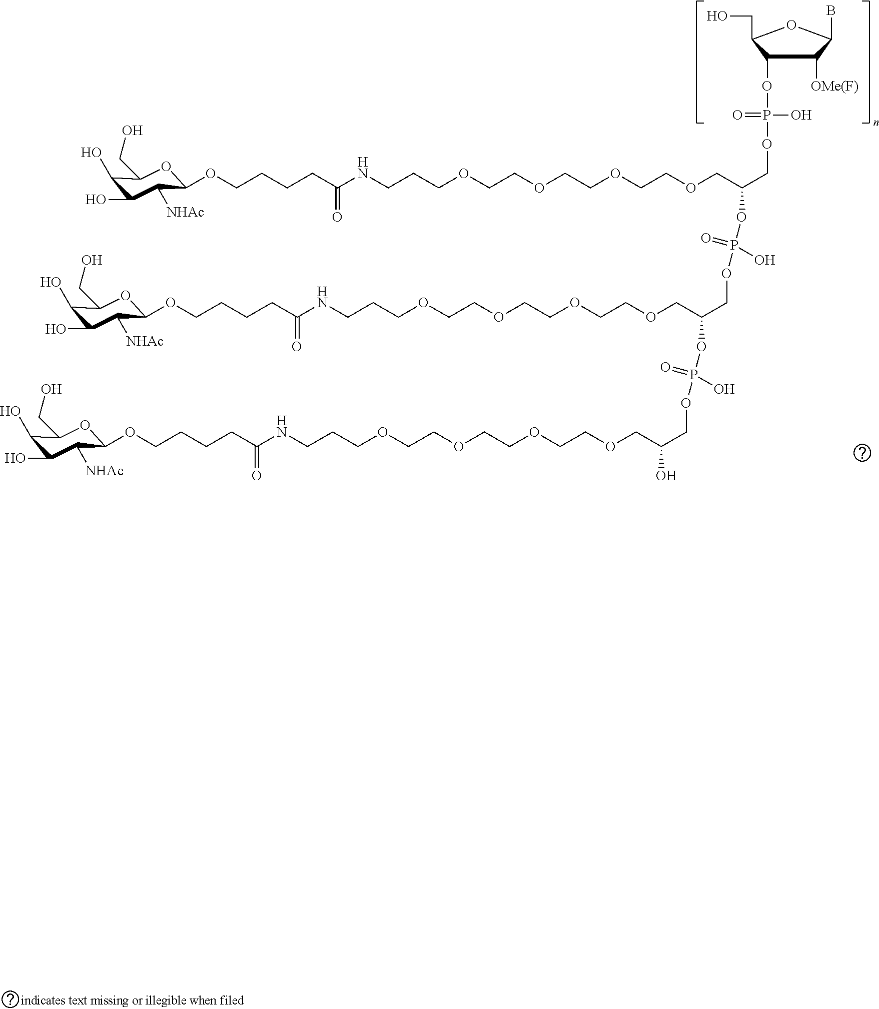

### Internucleoside Linkages

The nucleotide construct according to a second aspect, the disclosed embodiments may comprise one or more phosphorothioate or phosphorodithioate internucleotide linkages.

In certain embodiments, said nucleic acid construct may comprise 1 to 15 phosphorothioate or phosphorodithioate internucleotide linkages.

In certain embodiments, said nucleic acid construct may comprise one or more phosphorothioate or phosphorodithioate internucleotide linkages at one or more of the 5′ and/or 3′ regions of said first nucleic acid portion of (a), and/or said second nucleic acid portion of (b), and/or said third nucleic acid portion of (c), and/or said fourth nucleic acid portion of (d), and/or said 1 to 8 additional nucleic acid portions as defined previously herein, and/or said passenger nucleic acid portions as defined in previously herein.

In certain embodiments, said nucleic acid construct may comprise phosphorothioate or phosphorodithioate internucleotide linkages between at least two adjacent nucleotides of the nucleic acid linker portion as defined in previously herein.

In certain embodiments, said nucleic acid construct may comprise a phosphorothioate or phosphorodithioate internucleotide linkage between each adjacent nucleotide that is present in said nucleic acid linker portion.

In certain embodiments, said nucleic acid construct may comprises a phosphorothioate or phosphorodithioate internucleotide linkage linking: the first nucleic acid portion of (a) to the nucleic acid linker portion as defined in previously herein; and/or the second nucleic acid portion of (b) to the nucleic acid linker portion as defined previously herein; and/or the third nucleic acid portion of (c) to the nucleic acid linker portion as defined previously herein and/or the fourth nucleic acid portion of (d) to the nucleic acid linker portion as defined previously herein; and/or the 1 to 8 additional nucleic acid portions as defined previously herein to the nucleic acid linker portion as further defined previously herein; and/or the passenger nucleic acid portions as defined previously herein to the nucleic acid linker portion as further defined previously herein.

Modifications to siRNAs

The RNA molecules of the disclosed embodiments can be conjugated (e.g, at its 5′ or 3′ terminus of its sense or antisense strand) or unconjugated to another moiety (e.g, a non-nucleic cid moiety such as a peptide), an organic compound (e.g., a dye, cholesterol, or the like). Modifying RNA agents in this way may improve cellular uptake or enhance cellular targeting activities of the resulting RNA agent derivative as compared to the corresponding unconjugated RNA agent, are useful for tracing the RNA agent derivative in the cell or improve the stability of the RNA agent derivative compared to the corresponding unconjugated RNA agent.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphorodithioates, phosphoramidates, methyl phosphonates, chiral-methyl phosphonates, 2′-O-methyl ribonucleotides, 2′-Fluoro ribonucleotides, peptide-nucleic acids (PNAs) and unlocked nucleic acids (UNAs; see, e.g., Jensen et al. Nucleic Acids Symposium Series 52:133-4), and derivatives thereof.

As used herein, “nucleotide” is used as recognized in the art to include those with natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, nonnatural nucleotides, non-standard nucleotides and other, see, e.g., Usman and McSwiggen. Chapter 30: Catalytic RNA (Ribozymes) as Drugs. Annual Reports in Medicinal Chemistry. 30:285-94, 1995; Eckstein, et al., International PCT Publication No. WO 92/07065; Usman et al, International PCT Publication No. WO 93/15187; and Uhlman & Peyman. Antisense oligonucleotides: a new therapeutic principle. Chem. Rev. 4:543-84, 1990. There are several examples of modified nucleic acid bases known in the art as summarized by Limbach, et al. Summary: The modified nucleosides of RNA. Nucleic Acids Res. 22(12):2183-96, 1994. Examples of base modifications that can be introduced into nucleic acid molecules include, hypoxanthine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g 6-methyluridine), propyne, and others (Burgin, et al., Biochemistry 35:14090, 1996; Uhlman & Peyman, supra). A modified base indicates a nucleotide base other than adenine, guanine, cytosine, and uracil at the 1′ position or their equivalents.

As used herein, a modified nucleotide or modified residue refers to a nucleotide having one or more modifications, typically non-naturally occurring modifications, to the nucleoside, the base, pentose ring, or phosphate group, although modifications may include naturally occurring modifications produced by enzymes that modify nucleotides, such as methyltransferases. Non-naturally occurring modifications in nucleotides include those with 2′ modifications, e.g., 2′-methoxy (2′-OMe), 2′-methoxyethoxy, 2′-fluoro (2′-F), 2′-allyl, 2′-O-[2-(methylamino)-2-oxoethyl], 4′-thio, 4′-CH₂—O-2′-bridge, 4′-(CH₂)₂—O-2′-bridge, 2′-LNA or other bicyclic or “bridged” nucleoside analog, and 2′-O— (N-methylcarbamate) or those comprising base analogs.

As used herein, an amino modification means 2′—NH₂ or 2′-O—NH₂, which can be further modified, or be unmodified. Such modified groups are described, e.g., in Eckstein et al., U.S. Pat. No. 5,672,695 and Matulic-Adamic et al., U.S. Pat. No. 6,248,878. “Modified nucleotides” of the disclosed embodiments can also include nucleotide analogs as described above.

In reference to the nucleic acid molecules of the present disclosure, modifications may exist upon these agents in patterns on one or both strands of the double stranded ribonucleic acid (RNA). As used herein, modification at “alternating positions” indicates that every other nucleotide is a modified nucleotide or there is an unmodified nucleotide (e.g., an unmodified ribonucleotide) between every modified nucleotide over a defined length of a strand of the RNA (e.g., 5′-MNMNMN-3′; 3′-MNMNMN-5′; where M is a modified nucleotide and N is an unmodified nucleotide). The modification pattern starts from the first nucleotide position at either the 5′ or 3′ terminus according to a position numbering convention. The pattern of modified nucleotides at alternating positions may run the full length of the strand, but in certain embodiments includes at least 4, 6, 8, 10, 12, 14 nucleotides containing at least 2, 3, 4, 5, 6 or 7 modified nucleotides, respectively. Modifications with alternating pairs of positions indicates a pattern where two consecutive modified nucleotides are separated by two consecutive unmodified nucleotides over a defined length of a strand of the RNA (e.g., 5′-MMNNMMNNMMNN-3¹; 3′-MMNNMMNNMMNN-5′; where M is a modified nucleotide and N is an unmodified nucleotide). The modification pattern starts from the first nucleotide position at either the 5′ or 3′ terminus according to a position numbering convention such as those described herein. The pattern of modified nucleotides at alternating positions may run the full length of the strand, but advantageously includes at least 8, 12, 16, 20, 24, 28 nucleotides containing at least 4, 6, 8, 10, 12 or 14 modified nucleotides, respectively. These modification patterns are exemplary and the skilled artisan will recognize that additional patterns may be used.

In certain embodiments, the first and second oligonucleotide sequences of the siRNA exist on separate oligonucleotide strands that can be and typically are chemically synthesized. In some embodiments, both strands contain 19 nucleotides. These molecules may be completely complementary and have blunt ends, or they may have dTdT overhangs on one or both strands. In certain embodiments the siRNA strands have differing lengths, with one possessing a blunt end at the 3′ terminus of a first strand (sense strand) and a 3′ overhang at the 3′ terminus of a second strand (antisense strand). The siRNA can also contain one or more deoxyribonucleic acid (DNA) base substitutions.

In the nucleic acid construct according to a second aspect of the disclosed embodiments, at least one nucleotide of at least one of the following may be modified: the first nucleic acid portion of (a); and/or the second nucleic acid portion of (b); and/or the third nucleic acid portion of (c); and/or the fourth nucleic acid portion of (d); and/or to the extent present, the 1 to 8 additional nucleic acid portions as defined previously herein; and/or to the extent present, the passenger nucleic acid portions as defined previously herein; and/or to the extent present, the nucleic acid linker portion as further defined previously herein.

In an advantageous embodiment, one or more of the odd numbered nucleotides starting from the 5′ region of one of the following may be modified, and/or wherein one or more of the even numbered nucleotides starting from the 5′ region of one of the following are modified, wherein typically the modification of the even numbered nucleotides is a second modification that is different from the modification of odd numbered nucleotides: the first nucleic acid portion of (a); and/or
the second nucleic acid portion of (b); and/or the third nucleic acid portion of (c); and/or the fourth nucleic acid portion of (d); and/or to the extent present, the 1 to 8 additional nucleic acid portions as defined previously herein; and/or to the extent present, the passenger nucleic acid portions as defined previously herein.

In certain embodiments, one or more of the odd numbered nucleotides starting from the 3′ region of the third nucleic acid portion of (c) may be modified by a modification that is different from the modification of odd numbered nucleotides starting from the 5′ region of the first nucleic acid portion of (a); and/or one or more of the odd numbered nucleotides starting from the 3′ region of the fourth nucleic acid portion of (d) may be modified by a modification that is different from the modification of odd numbered nucleotides starting from the 5′ region of the second nucleic acid portion of (b); and/or one or more of the odd numbered nucleotides starting from the 3′ region of the passenger nucleic acid portions as defined previously herein, to the extent present, may be modified by a modification that is different from the modification of odd numbered nucleotides starting from the 5′ region of the 1 to 8 additional nucleic acid portions as defined previously herein; and/or wherein one or more of the nucleotides of a nucleic acid linker portion as further defined previously herein, to the extent present, may be modified by a modification that (i) is different from the modification of an adjacent nucleotide of the 3′ region of the first nucleic acid portion of (a); and/or (ii) is different from the modification of an adjacent nucleotide of the 3′ region of the second nucleic acid portion of (b); and/or is different from the modification of an adjacent nucleotide of the 3′ region of the 1 to 8 additional nucleic acid portions, to the extent present, as defined previously herein.

In certain embodiments, one or more of the even numbered nucleotides starting from the 3′ region of: (i) the third nucleic acid portion of (c), and/or (ii) the fourth nucleic acid portion of (d), and/or (iii) said passenger nucleic acid portions as defined previously herein, to the extent present, may be modified by a modification that is different from the modification of odd numbered nucleotides starting from the 3′ region of these respective portions.

In certain embodiments, at least one or more of the modified even numbered nucleotides of (i) the first nucleic acid portion of (a), and/or (ii) the second nucleic acid portion of (b), and/or (iii), to the extent present, the 1 to 8 additional nucleic acid portions as defined previously herein, may be adjacent to at least one or more differently modified odd numbered nucleotides of these respective portions.

In certain embodiments, at least one or more of the modified even numbered nucleotides of (i) the third nucleic acid portion of (c), and/or (ii) the fourth nucleic acid portion of (d), and/or (iii), to the extent present, the passenger nucleic acid portions as defined previously herein, may be adjacent to at least one or more differently modified odd numbered nucleotides of these respective portions.

In certain embodiments, a plurality of adjacent nucleotides of (i) the first nucleic acid portion of (a), and/or (ii) the second nucleic acid portion of (b), and/or (iii), to the extent present, the 1 to 8 additional nucleic acid portions as defined previously herein, may be modified by a common modification.

In certain embodiments, a plurality of adjacent nucleotides of (i) the third nucleic acid portion of (c), and/or (ii) the fourth nucleic acid portion of (d), and/or (iii), to the extent present, the passenger nucleic acid portions as defined previously herein, may be modified by a common modification.

In certain embodiments, said plurality of adjacent commonly modified nucleotides may be 2 to 4 adjacent nucleotides, advantageously 3 or 4 adjacent nucleotides.

In certain embodiments, said plurality of adjacent commonly modified nucleotides may be located in the 5′ region of (i) the third nucleic acid portion of (c), and/or (ii) the fourth nucleic acid portion of (d), and/or (iii), to the extent present, the passenger nucleic acid portions previously herein.

In certain embodiments, a plurality of adjacent commonly modified nucleotides may be located in the nucleic acid linker portion as further defined previously herein.

In certain embodiments, the one or more of the modified nucleotides of first nucleic acid portion of (a) may not have a common modification present in the corresponding nucleotide of the third nucleic acid portion of (c) of the first duplex region; and/or one or more of the modified nucleotides of second nucleic acid portion of (b) may not have a common modification present in the corresponding nucleotide of the fourth nucleic acid portion of (d) of the second duplex
region; and/or one or more of the modified nucleotides of the 1 to 8 additional nucleic acid portions, to the extent present, as defined previously herein, may not have a common modification present in the corresponding nucleotide of the corresponding passenger nucleic acid portions of the respective duplex regions.

In certain embodiments, the one or more of the modified nucleotides of the first nucleic acid portion of (a) may be shifted by at least one nucleotide relative to a commonly modified nucleotide of the third nucleic acid portion of (c); and/or one or more of the modified nucleotides of the second nucleic acid portion of (b) may be shifted by at least one nucleotide relative to a commonly modified nucleotide of the fourth nucleic acid portion of (d); and/or one or more of the modified nucleotides of the 1 to 8 additional nucleic acid portions, to the extent present, as defined previously herein may be shifted by at least one nucleotide relative to a commonly modified nucleotide of the passenger nucleic acid portions, to the extent present, as defined previously herein.

In certain embodiments, the modification and/or modifications may be each and individually sugar, phosphate, or base modifications.

In certain embodiments, the modification may be selected from nucleotides with 2′ modified sugars; conformationally restricted nucleotides (CRN) sugar such as locked nucleic acid (LNA), (S)-constrained ethyl bicyclic nucleic acid, and constrained ethyl (cEt), tricyclo-DNA; morpholino, unlocked nucleic acid (UNA), glycol nucleic acid (GNA), D-hexitol nucleic acid (HNA), and cyclohexene nucleic acid (CeNA). In advantageous embodiments, wherein said 2′ modified sugar may be selected from 2′-O-alkyl modified sugar, 2′-O-methyl modified sugar, 2′-O-methoxy ethyl modified sugar, 2′-O-allyl modified sugar, 2′-C-allyl modified sugar, 2′-deoxy modified sugar such as 2′-deoxy ribose, 2′-F modified sugar, 2′-arabino-fluoro modified sugar, 2′-O-benzyl modified sugar, 2′-amino modified sugar, and 2′-O-methyl-4-pyridine modified sugar.

In certain embodiments, the base modification may be any one of an abasic nucleotide and a non-natural base comprising nucleotide.

In certain embodiments, at least one modification may be a 2′-O-methyl modification in a ribose moiety.

In certain embodiments, at least one modification may be a 2′-F modification in a ribose moiety.

In certain embodiments, the nucleotides at any of positions 2 and 14 downstream from the first nucleotide of the 5′ region of (i) the first nucleic acid portion of (a); and/or (ii) the second nucleic acid portion of (b); and/or (iii), to the extent present, the 1 to 8 additional nucleic acid portions as defined previously herein; may not contain 2′-O-methyl modifications in ribose moieties.

In certain embodiments, one, two or all three nucleotides of (i) the third nucleic acid portion of (c); and/or (ii) the fourth nucleic acid portion of (d); and/or (iii), to the extent present, said passenger nucleic acid portions as defined previously herein; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii) the 1 to 8 additional nucleic acid portions, to the extent present, as defined previously herein; may not contain 2′-O-methyl modifications in ribose moieties.

In certain embodiments, the nucleotides at any of positions 2 and 14 downstream from the first of (i) the first nucleic acid portion of (a); and/or (ii) the second nucleic acid portion of (b); and/or (iii), to the extent present, the 1 to 8 additional nucleic acid portions as defined previously herein; may contain 2′-F modifications in ribose moieties.

In certain embodiments, one, two or all three nucleotides of (i) the third nucleic acid portion of (c); and or (ii) the fourth nucleic acid portion of (d); and/or (iii), to the extent present, said passenger nucleic acid portions as defined previously herein; that respectively correspond in position to any of the nucleotides at any of positions 11 to 13 downstream from the first nucleotide of the 5′ region of (i) the first nucleic acid portion of (a); and/or (ii) the second nucleic acid portion of (b); and/or (iii), to the extent present, the 1 to 8 additional nucleic acid portions as defined previously herein; may contain 2′-F modifications in ribose moieties.

In certain embodiments, all remaining nucleotides may contain either 2′-O-methyl modifications or 2′-F modifications in ribose moieties, advantageously with the exception of the unmodified nucleotide(s) in accordance with the labile linkage defined herein. Advantageously, said remaining nucleotides may contain 2′-O-methyl modifications in ribose moieties.

In certain embodiments, said one or more, advantageously one, unmodified nucleotide represents any of the nucleotides of the nucleic acid linker portion as further defined previously herein, advantageously the nucleotide of the nucleic acid linker portion as further defined
previously herein that is adjacent to (i) the third nucleic acid portion of (c); and or (ii) the fourth nucleic acid portion of (d); and/or (iii), to the extent present, said passenger nucleic acid portions as defined previously herein.

In certain embodiments, the 3′ terminal positions of said first and said third nucleic acid portions may be replaced with an unmodified nucleotide.

In certain embodiments, the nucleic acid construct may comprise at least one vinylphosphonate modification, such as at least one vinylphosphonate modification in the 5′ region of (i) the first nucleic acid portion of (a); and/or (ii) the second nucleic acid portion of (b); and/or (iii), to the extent present, the 1 to 8 additional nucleic acid portions as defined previously herein.

In certain embodiments, one or more nucleotides of the first nucleic acid portion of (a); and/or the second nucleic acid portion of (b); and/or the third nucleic acid portion of (c); and/or the fourth nucleic acid portion of (d); and/or to the extent present, the 1 to 8 additional nucleic acid portions as defined previously herein; and/or to the extent present, the passenger nucleic acid portions as defined previously herein; may be an inverted an inverted nucleotide and may be attached to the adjacent nucleotide via the 3′ carbon of the nucleotide and the 3′ carbon of the adjacent nucleotide, and/or may be an inverted nucleotide and may be attached to the adjacent nucleotide via the 5′ carbon of the nucleotide and the 5′ carbon of the adjacent nucleotide.

In certain embodiments, the inverted nucleotide may be attached to the adjacent nucleotide via a phosphate group by way of a phosphodiester linkage; or may be attached to the adjacent nucleotide via a phosphorothioate group; or may be attached to the adjacent nucleotide via a phosphorodithioate group.

In certain embodiments, the modifications among strands within the constructs include alternating modification pattern, advantageously with odd-numbered nucleotides being fluorosubstituted and even-numbered nucleotides being-OMe substituted.

Compositions and Pharmaceutical Compositions Including muRNA Oligomeric Constructs

According to a second aspect, the disclosed embodiments are directed to a composition comprising a nucleic acid construct according to the first aspect, and a physiologically acceptable excipient.

According to a third aspect, the disclosed embodiments are directed to a pharmaceutical composition comprising a nucleic acid construct according to the first aspect.

Said pharmaceutical composition may further comprise a pharmaceutically acceptable excipient, diluent, antioxidant, and/or preservative.

Said oligomeric compound according to the first aspect and/or the construct according to the second aspect may be the only pharmaceutically active agent(s).

Alternatively, said pharmaceutical composition furthermore comprises one or more further pharmaceutically active agents. Advantageously, said further pharmaceutically active agents are selected from:
Vascepa; Vupanorsen; statins such as Rosuvastatin and Simvastatin; fibrates such fenofibrate; LDL-cholesterol lowering compounds such as statins and ezetimib, and/or agents which decrease hypertension, wherein said further pharmaceutically active agent(s) is/are optionally selected from the group consisting of a diuretic, an angiotensin converting enzyme (ACE) inhibitor, an angiotensin II receptor antagonist, a beta-blocker, a vasodilator, a calcium channel blocker, an aldosterone antagonist, an alpha2-agonist, a renin inhibitor, an alpha-blocker, a peripheral acting adrenergic agent, a selective DI receptor partial agonist, a nonselective alpha-adrenergic antagonist, a synthetic, a steroidal antimineralocorticoid agent; a combination of any of the foregoing; and a hypertension therapeutic agent formulated as a combination of agents, more optionally an angiotensin II receptor antagonist selected from the group consisting of losartan, valsartan, olmesartan, eprosartan, and azilsartan.
   Determination of Efficacy of the siRNA Molecules

Depending on the particular target, one or more siRNA sequences targeting one or more proteins, and the dose of the nanoparticle composition delivers, partial or complete loss of function for the targeted proteins may be observed. A reduction or loss of RNA levels or targeted protein expression of the targeted proteins or encoded polypeptide expression in at least 50%, 60%, 70%, 80%, 90%, 95% or 99% or more of targeted cells is exemplary. Inhibition of the target's protein levels or expression refers to the absence (or observable decrease) in the level
of targets' RNA or RNA-encoded protein. Specificity refers to the ability to inhibit the targeted protein's RNA without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Inhibition of targets' RNA sequence(s) by the siRNA agents of the disclosed embodiments also can be measured based upon the effect of administration of such siRNA agents upon development/progression of a target protein's associated disease or disorder, e.g., tumor formation, growth, metastasis, etc., either in vivo or in vitro. Treatment and/or reductions in tumor or cancer cell levels can include halting or reduction of growth of tumor or cancer cell levels or reductions of, e.g., 10%, 20%, 30%, 40%, 50%, 60%, 70%, 80%, 90%, 95% or 99% or more, and can also be measured in logarithmic terms, e.g., 10-fold, 100-fold, 1000-fold, 10⁴-fold, 10²-fold, 10⁶-fold, or 10⁷-fold reduction in cancer cell levels could be achieved via administration of the nanoparticle composition to cells, a tissue, or a subject. The subject may be a mammal, such as a human.

### Determination of Dosage and Toxicity

Toxicity and therapeutic efficacy of the compositions may be determined by standard pharmaceutical procedures in cell cultures or experimental animals, e.g., by determining the LDso (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds advantageously exhibit high therapeutic indices.

Data from cell culture assays and animal studies can be used in formulating a range of doses for use in humans. The dosage of the compositions advantageously is within a range of circulating concentrations that include the ED50 with little or no toxicity. The dosage may vary within this range depending upon the dosage form employed and the route of administration utilized. For the compositions described herein, a therapeutically effective dose can be estimated initially from cell culture assays. A dose may be formulated in animal models to achieve a circulating plasma concentration range that includes the IC50 (i.e., the concentration of the
composition which achieves a half-maximal inhibition of symptoms) as determined in cell culture. Such information can be used to more accurately determine useful doses in humans. Levels in plasma may be measured, for example, by high performance liquid chromatography (HPLC).

A therapeutically effective amount of a composition as described herein can be in the range of approximately 1 μg to 1000 mg. For example, 10, 30, 100, or 1000 μg, or 10, 30, 100, or 1000 ng, or 10, 30, 100, or 1000 μg, or 10, 30, 100, or 1000 mg, or 1-5 g of the compositions can be administered. In general, a suitable dosage unit of the compositions described herein will be in the range of 0.001 to 0.25 milligrams per kilogram body weight of the recipient per day, or in the range of 0.01 to 20 micrograms per kilogram body weight per day, or in the range of 0.001 to 5 micrograms per kilogram of body weight per day, or in the range of 1 to 500 nanograms per kilogram of body weight per day, or in the range of 0.01 to 10 micrograms per kilogram body weight per day, or in the range of 0.10 to 5 micrograms per kilogram body weight per day, or in the range of 0.1 to 2.5 micrograms per kilogram body weight per day. The pharmaceutical composition can be administered once daily, or may be dosed in dosage units containing two, three, four, five, six or more sub-doses administered at appropriate intervals throughout the day. In that case, the siRNA contained in each sub-dose must be correspondingly smaller in order to achieve the total daily dosage unit. The dosage unit can also be compounded for a single dose over several days, e.g., using a conventional sustained release formulation which provides sustained and consistent release of the siRNA over a several day period. Sustained release formulations are well known in the art. In this embodiment, the dosage unit contains a corresponding multiple of the daily dose. Regardless of the formulation, the pharmaceutical composition must contain siRNA in a quantity sufficient to inhibit expression of the target gene in the animal or human being treated. The composition can be compounded in such a way that the sum of the multiple units of siRNA together contain a sufficient dose.

The compositions may be administered once, one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a
therapeutically effective amount of a composition as described herein may include a single treatment or, advantageously, can include a series of treatments.

As used herein, a pharmacologically or therapeutically effective amount refers to that amount of an siRNA composition effective to produce the intended pharmacological, therapeutic or preventive result. The phrases “pharmacologically effective amount” and “therapeutically effective amount” or “effective amount” refer to that amount of the composition effective to produce the intended pharmacological, therapeutic or preventive result. For example, if a given clinical treatment is considered effective when there is at least a 30% reduction in a measurable parameter associated with a disease or disorder, a therapeutically effective amount of a drug for the treatment of that disease or disorder is the amount necessary to affect at least a 30% reduction in that parameter.

Suitably formulated pharmaceutical compositions as described herein may be administered by means known in the art such as by parenteral routes, including intravenous, intramuscular, intraperitoneal, subcutaneous, transdermal, airway (aerosol), rectal, vaginal and topical (including buccal and sublingual) administration. Advantageously, the pharmaceutical compositions are administered by intravenous or intraparenteral infusion or injection.

### Pharmaceutical Compositions and Methods of Administration

The nanoparticle compositions may be further formulated as a pharmaceutical composition using methods that are well known in the art. The composition may be formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, trehalose, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The compositions may also be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard
techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

### Methods of Treatment

The compositions may be administered as described above and, advantageously, may be delivered systemically or intratum orally. The compositions may be administered as a monotherapy, i.e., in the absence of another treatment, or may be administered as part of a combination regimen that includes one or more additional medications. Advantageously, the compositions are used as part of a combination regimen that includes an effective amount of at least one additional chemotherapy drug, as described below.

Diseases to be Treated by muRNA Oligomeric Compounds and Further Uses

According to a fourth aspect, the disclosed embodiments are directed to the nucleic acid construct according to first aspect, for use in human or veterinary medicine or therapy.

According to a fifth aspect, the disclosed embodiments are directed to the nucleic acid construct according to the first aspect, for use in a method of treating, ameliorating and/or preventing a disease or disorder.

Said disease or disorder is a disease or disorder associated with APOC3 and/or AGT or a disease or disorder requiring reduction of APOC3 and/or AGT expression.

In particular, said disease or disorder is selected from the group consisting of an AP0C3-associated disease or disorder, or a disease or disorder requiring reduction of APOC3 expression levels, said disease or disorder advantageously being selected from dyslipidemia including mixed dyslipidemia; hyperchylomicronemia including familial hyperchylomicronemia; hypertriglyceridemia, advantageously severe hypertriglyceridemia and/or hypertriglyceridemia with blood triglyceride levels above 500 mg/dl; inflammation including low-grade inflammation; atherosclerosis; atherosclerotic cardiovascular diseases (ASCVD) including major adverse cardiovascular events (MACE) such as myocardial infarction, stroke and peripheral arterial disease; pancreatitis including acute pancreatitis; and/or
high blood pressure, hypertension, borderline hypertension, primary hypertension, secondary hypertension isolated systolic or diastolic hypertension, pregnancy-associated hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, vasculopathy, diabetic nephropathy, diabetic retinopathy, chronic heart failure, cardiomyopathy, diabetic cardiac myopathy, nocturnal hypotension, glomerulosclerosis, coarctation of the aorta, aortic aneurism, ventricular fibrosis, heart failure, myocardial infarction, angina, stroke, renal disease, renal failure, systemic sclerosis, intrauterine growth restriction (IUGR), fetal growth restriction, obesity, liver steatosis/fatty liver, nonalcoholic Steatohepatitis (NASH), non-alcoholic fatty liver disease (NAFLD); glucose intolerance, type 2 diabetes, and metabolic syndrome.

According to a sixth aspect, the disclosed embodiments are directed to a method of treating a disease or disorder comprising administration the nucleic acid construct according to the first aspect, to an individual in need of treatment.

The nucleic acid construct may be administered subcutaneously or intravenously to the individual.

According to a seventh aspect, the disclosed embodiments are directed to a use of a nucleic acid construct according to the first aspect, for use in research as a gene function analysis tool.

According to an eighth aspect, the disclosed embodiments are directed to a use of the nucleic acid construct according to the first aspect in the manufacture of a medicament for a treatment of a disease or disorder.

It should also be noted that the scope of the disclosed embodiments extends to sequences that correspond to those disclosed above, and wherein the 5′ terminal nucleoside of the antisense (guide) strand can include any nucleobase that can be present in an RNA molecule, in other words can be any of adenine (A), uracil (U), guanine (G) or cytosine (C). Additionally, the scope of the disclosed embodiments extends to sequences that correspond to those disclosed above, and wherein the 3′ terminal nucleoside of the sense (passenger) strand can include any nucleobase that can be present in an RNA molecule, in other words can be any of adenine (A), uracil (U), guanine (G) or cytosine (C), advantageously however a nucleobase that is complementary to the 5′ nucleobase of the antisense (guide) strand. Specific examples include the sequences of SEQ ID NOs: 142 and 143, 144 and 145, and 146 and 147, where these positions have been modified as compared to the sequences of SEQ ID NOs: 103 and 131, 132 and 133, and 134 and 135, respectively.

While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the Examples described above may be combined with aspects of any of the other Examples described to form further Examples.

It will be understood that the above description of an advantageous embodiment is given by way of example only and that various modifications may be made by those skilled in the art. What has been described above includes Examples of one or more embodiments. It is, of course, not possible to describe every conceivable modification and alteration of the above compounds, compositions or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

### EXAMPLES

The following Examples illustrate certain embodiments of the present disclosure and are not limiting. Moreover, where specific embodiments are provided, the inventors have contemplated generic application of those specific embodiments. For example, disclosure of an oligonucleotide having a particular motif or modification patterns provides reasonable support for additional oligonucleotides having the same or similar motif or modification patterns.

The syntheses of the RNAi constructs according to the disclosed embodiments will be carried out using synthesis methods known to the person skilled in the art, such as synthesis methods disclosed in https://en.wikipedia.org/wiki/Oligonucleotide synthesis, wherein the methods disclosed on this website are incorporated by reference herein in their entirety. The only difference to the synthesis method disclosed in this reference is that GalNAc phosphoramidite immobilized on a support is used in the synthesis method during the first synthesis step.

### Example 1: In Vitro Assay

### Materials and Methods

### Cell Culture

Human primary hepatocytes (5 donor pooled-Sekisui XenoTech, HPCH05+) were thawed immediately prior to experimentation and cultured in 1× complete Williams medium (Gibco, A1217601) supplemented with Hepatocytes plating supplement pack (Gibco, CM3000). FBS concentration was modified from manufacture recipe to a final 2.5% (as opposed to 5%) for compound stability.

### Compounds Preparation

A seven step, five-fold dilution series of each compound was prepared in basal WEM from 2 μM to 0.000128 μM.

### Transfection

On the day of transfection, primary human hepatocytes were thawed in 45 mL of human OptiThaw (Sekisui XenoTech, K8000) and centrifuged down at 200 g for 5 minutes. Cells were resuspended in 2× complete WEM and counted. Cells were then plated in 50 μL of 2× complete WEM at 25,000 cells per well on 96 well type 1 rat tail Collagen plates and allowed to rest and attach for four hours before transfection. After rest, 50 μL of each dilution was added to respective triplicates of the plated hepatocytes for a final dilution series of 1 μM down to 0.000064 μM in a volume of 100 uL 1× complete WEM.

qPCR

72 hours post transfection, cells were harvested and RNA isolated using the PureLink Pro 96 total RNA Purification Kit (ThermoFisher, 12173011A) according to the manufacturer protocol. Harvested RNA was assayed for ApoC3 and AGT expression via Taqman qPCR using the Luna Universal Probe One-Step RT-qPCR Kit (NEB, E3006). A qPCR assay was performed for each sample using a ApoC3 TaqMan probe set (Hs00906501_gl-FAM) or AGT Tagman probe (Hs01 586213_ml-FAM) multiplexed with a common GAPDH VIC probe (ThermoFisher, 4326317E). Thermocycling and data acquisition was performed with an Applied Biosystems QuantStudio 3/5 Real-Time PCR System.

### Results

Results are shown in FIGS. 1 and 2 as well as in Tables 3 and 4 below.

TABLE 3
AGT knock-down at 1000 nM and IC50 (in nM)

| SEQ | | KD % at | IC50 |
|---|---|---|---|
| ID Nos. | Construct Name | 1000 nM | (nM) |
| 164-165 | APO28-AGT52A | 82.96 | 1.955 |
| 148-149 | APO28-AGT27 | 88.82 | 1.867 |
| 160-161 | APO28-AGT27A | 89.09 | 0.553 |
| 162-163 | APO28-AGT62A | 60.76 | 13.270 |

TABLE 4
APOC3 knock-down at 1000 nM and IC50 (in nM)

| SEQ | | KD % at | IC50 |
|---|---|---|---|
| ID Nos. | Construct Name | 1000 nM | (nM) |
| 164-165 | APO28-AGT52A | 87.10 | 0.953 |
| 148-149 | APO28-AGT27 | 95.50 | 0.753 |
| 160-161 | APO28-AGT27A | 95.82 | 0.432 |
| 162-163 | APO28-AGT62A | 94.85 | 0.477 |

The tested double-targeting muRNAs perform surprisingly outstandingly and inhibit either target with an IC50 value in a picomolar to single-digit (in one case double-digit) nanomolar range.

### Example 2: Dose Response Study Evaluating Angiotensinogen-Apolipoprotein C3 Combination (AGT-APOC3; muRNA) in Humanized Liver-uPA-SCID Mice Model Materials and Methods

### 1. Study Objective(s)

The objective of this non-GLP study is to evaluate, in humanized liver-uPA-SCID mice: i. The dose response of GalNAc conjugated human Angiotensinogen (AGT) targeting mxRNA constructs ii. The dose response of GalNAc conjugated human dual targeting (AGT and APOC3)

muRNA Constructs

The compound(s) will be administered subcutaneously, and the mice will be survived for up to 14 days.

Prior to necropsy, blood will be collected for plasma samples. At necropsy, 3 liver biopsies (2 mm) per animal will be preserved in separate vials in RNA/rz/er, flash frozen, and stored at −80° C. Three more liver biopsies (2 mm) will be taken, flash frozen in the same vial, and stored at −80° C. The remaining liver will be flash frozen and stored at −80° C.

### 2. Test System Information

2.1. Animal Test
2.1.1. Common Name: Mouse
2.1.2. Breed/Class: Rodent-humanized liver-uP A-SCID mice
2.1.3. Number of Animals (by gender): 60 Male all naive
2.1.4. Age Range: 14-19 weeks for mice,
2.1.5. Weight Range: Approx. 20 grams for all mice

### 3. Study Design

3.1. Design Details

This study will have one type of mice, 60 humanized liver-uP A-SCID mice. Animals will be grouped by treatment type, dosage, and survival period. Each animal will be treated by subcutaneous injection of test material. (Note: that the injection must be given subcutaneously. The test articles will not be functional if the subcutaneous site is missed, and injection is given within the muscular region or test articles are injected into the vein/bloodstream).
Group 1A will have four animals and receive a control dose of PBS.
Group 2A, 2B and 2C will have four/five animals and receive a single dose of (AGT27A) at 5 mg/kg, 10 mg/kg and 30 mg/kg, respectively.
Group 3A, 3B and 3C will have four/five animals and receive a single dose of (AGT52A) at 5 mg/kg, 10 mg/kg, and 30 mg/kg, respectively.
Group 4A, 4B and 4C will have four/five animals and receive a single dose of (AGT27A-A28) at 5m/kg, 10 mg/kg, and 30 mg/kg, respectively.
Group 5A, 5B and 5C will have four/five animals and receive a single dose of (AGT52A-A28) at 5m/kg, 10 mg/kg, and 30 mg/kg, respectively.

Animals will be survived for 14 days. See Study Table 5 for details.

Prior to necropsy, the animals will be deeply anesthetized, and a terminal blood draw will be performed through the vena cava. Blood volume will be collected in a plasma separation tube.

After separation the plasma sample will be split evenly into two labeled vials, flash frozen, and stored at −80° C.

Note: plasma will be used to measure protein, caution should be taken to avoid hemolysis or clot formation. At necropsy, three 2 mm biopsy punches will be taken from the left, middle and right liver lobes, placed in separate vials, soaked in RNA later for 15 minutes, flash frozen and stored at −80° C. Another three 2 mm liver biopsies from the left, middle and right liver lobes will be placed into one vial, flash frozen and stored at −80° C. The rest of the liver will be flash frozen and stored in 10 ml conical tubes at −80° C.

TABLE 5
Study Table

| | | Treatment | | | |
|---|---|---|---|---|---|
| | Number | Subcutaneous | | | |
| | of | Injection | Survival | | Pre-Euthanasia |
| Group | Animals | Day 0 | Days | Blood | and Necropsy |
| 1A | 4 | Control (PBS) | 14 | Blood | Pre-Euthanasia: |
| | | | | collected | Plasma and |
| 2A | 4 | AGT27A | 14 | for plasma. | collection. |
| | | (5 mg/kg) | | Plasma | Necropsy: |
| | | (SEQ ID No. 166) | | will be | 2 mm biopsy of |
| 2B | 5 | AGT27A | 14 | evenly | left, middle and |
| | | (10 mg/kg) | | separated | right liver lobes |
| 2C | 5 | AGT27A | 14 | into two | in separate vials, |
| | | (30 mg/kg) | | labeled | in RNAlater for |
| 3A | 4 | AGT52A | 14 | vials. | 15 min, flash |
| | | (5 mg/kg) | | | freeze then stored |
| 3B | 5 | AGT52A | 14 | | at −80° C. |
| | | (10 mg/kg) | | | 2 mm biopsy of |
| 30 | 5 | AGT52A | 14 | | left, middle and |
| | | (30 mg/kg) | | | right liver all in |
| 4A | 4 | AGT27A-A28 | 14 | | one vial, flash |
| | | (5 mg/kg) | | | freeze then stored |
| 4B | 5 | AGT27A-A28 | 14 | | at −80° C. |
| | | (10 mg/kg) | | | |
| 4C | 5 | AGT27A-A28 | 14 | | |
| | | (30 mg/kg) | | | |
| 5A | 4 | AGT52A-A28 | 14 | | |
| | | (5 mg/kg) | | | |
| 5B | 5 | AGT52A-A28 | 14 | | |
| | | (10 mg/kg) | | | |
| 5C | 5 | AGT52A-A28 | 14 | | |
| | | (30 mg/kg) | | | |
| Total | 60 | | | | |
| | | Treatment | | | |
| | Number | Subcutaneous | | | |
| | of | Injection | Survival | | Pre-Euthanasia |
| Group | Animals | Day 0 | Days | Blood | and Necropsy |
| | | | | | Rest of liver, |
| | | | | | flash freeze then |
| | | | | | stored at −80° C. |

AGT27A is SEQ ID No. 166:
[5Phos][mA][Ps][fU][Ps][mA][fG][mA][fA][mG][fA][mA][fA][mA][fG][mG][fU][Ps][mG]
[Ps][fG][Ps][mG][Ps][fA][Ps][mG][Ps][mA][fC][fC][fU][mU][fU][mU][fC][mU][fU][mC][fU]
[Ps][mA][Ps][fU][Ps][3xGalNac]

### 4. Test Article and Ancillary Material Information

4.1. Test Drug 1:4.
1.1. Identification: AGT27A
4.1.2. Manufacturer: Sirnaomics
4.1.3. Description: GalNAc-mxRNA targeting human angiotensinogen
(AGT) mRNA
4.1.4. Lot/Batch Number: Will be recorded on study materials form.
4.1.5. Expiration Date: Will be recorded on study materials form.
4.1.6. Storage Temperature: 4° C.
4.1.7. Bio-Hazard Status: None
4.1.8. SDS*: TBD
4.1.9. Appearance: Clear Liquid
4.1.10. Dose Information: See Study Table 5
4.1.11. Residual Test Article Storage: None
4.2. Test Drug 2:4.
2.1. Identification: AGT52A
4.2.2. Manufacturer: Sirnaomics
4.2.3. Description: GalNAc-mxRNA targeting human angiotensinogen
(AGT) mRNA
4.2.4. Lot/Batch Number: Will be recorded on study materials form.
4.2.5. Expiration Date: Will be recorded on study materials form.
4.2.6. Storage Temperature: 4° C.
4.2.7. Bio-Hazard Status: None
4.2.8. SDS*: TBD
4.2.9. Appearance: Clear Liquid
4.2.10. Dose Information: See Study Table 5
4.2.11. Residual Test Article Storage: None
4.3. Test Drug 3:
4.3.1. Identification: AGT27A-A28
4.3.2. Manufacturer: Sirnaomics
4.3.3. Description: GalNAc-muRNA targeting human angiotensinogen
(AGT) and apolipoprotein C3 (APOC3) mRNA
4.3.4. Lot/Batch Number: Will be recorded on study materials form.
4.3.5. Expiration Date: Will be recorded on study materials form.
4.3.6. Storage Temperature: 4° C.
4.3.7. Bio-Hazard Status: None
4.3.8. SDS*: TBD
4.3.9. Appearance: Clear Liquid
4.3.10. Dose Information: See Study Table 5
4.3.11. Residual Test Article Storage: None
4.4. Test Drug 4:4.
4.1. Identification: AGT52A-A28
4.4.2. Manufacturer: Sirnaomics
4.4.3. Description: GalNAc-muRNA targeting human angiotensinogen
(AGT) and apolipoprotein C3 (APOC3) mRNA
4.4.4. Lot/Batch Number: Will be recorded on study materials form.
4.4.5. Expiration Date: Will be recorded on study materials form.
4.4.6. Storage Temperature: 4° C.
4.4.7. Bio-Hazard Status: None
4.4.8. SDS*: TBD
4.4.9. Appearance: Clear Liquid
4.4.10. Dose Information: See Study Table 5
4.4.11. Residual Test Article Storage: None

### 5. Necropsy and Explant Procedure

Note: Tissue samples will bes taken using separate tools for each individual collection.

Tissue harvesting tools will be changed for each tissue sample to prevent cross contamination.

A 2 mm biopsy punch will be taken from the left, middle and right liver lobes. Place biopsy samples into separate 2 ml Eppendorf tubes, with 1.5 ml RNA/ater and let soak for 15 minutes, flash freeze then store at −80° C. Three more 2 mm biopsy samples will be taken of the left, middle and right liver lobes all placed together into one 2 ml Eppendorf tubes, flash freeze then store at −80° C. Remaining liver will be flash frozen and stored in 10 ml conical tubes at −80° C.

### RESULTS

Results are shown in FIGS. 3 to 8. Outstanding knock-down, both at the mRNA as well as at the protein level could be demonstrated.

1. A nucleic acid construct comprising or consisting of:
(a) a first nucleic acid portion that is at least partially complementary to at least a first portion of an RNA which is transcribed from an APOC3 gene;
(b) a second nucleic acid portion that is at least partially complementary to at least a second portion of an RNA which is transcribed from an AGT gene;
(c) a third nucleic acid portion that is at least partially complementary to said first nucleic acid portion of (a), so as to form a first nucleic acid duplex region therewith;
(d) a fourth nucleic acid portion that is at least partially complementary to said second nucleic acid portion of (b), so as to form a second nucleic acid duplex region therewith,
said construct is designed such that subsequent to in vivo administration said construct disassembles to yield at least first and second discrete nucleic acid targeting molecules that respectively target said RNA portions transcribed from said target genes of (a) and (b); whereby
(i) said first nucleic acid targeting molecule is capable of modulating expression of said target gene of (a), and comprises, or is derived from, at least said first nucleic acid portion of (a),
and (ii) said second nucleic acid targeting molecule is capable of modulating expression of said target gene of (b), and comprises, or is derived from, said second nucleic acid portion of (b),
said construct is designed to disassemble such that said first and second discrete nucleic acid targeting molecules are respectively processed by independent RNAi-induced silencing complexes.

2. (canceled)

3. (canceled)

4. The construct according to claim 1, which further comprises at least one labile functionality such that subsequent to in vivo administration said construct is cleaved so as to yield said at least first and second discrete nucleic acid targeting molecules,
said labile functionality comprises one or more unmodified nucleotides,
said one or more unmodified nucleotides of said labile functionality represent one or more cleavage positions within said construct whereby subsequent to in vivo administration said construct is cleaved at said one or more cleavage positions so as to yield said at least first and second discrete nucleic acid targeting molecules,
said cleavage positions are respectively located within the construct so that after cleavage said first discrete nucleic acid targeting molecule comprises, or is derived from, said first nucleic acid duplex region, and said second discrete nucleic acid targeting molecule comprises, or is derived from, said second nucleic acid duplex region,
said first discrete nucleic acid targeting molecule comprises or consists of said first nucleic acid portion of (a) and said third nucleic acid portion of (c), and/or said second discrete nucleic acid targeting molecule comprises or consists of said second nucleic acid portion of (b) and said fourth nucleic acid portion of (d).

5. (canceled)

6. (canceled)

7. (canceled)

8. (canceled)

9. The construct according to claim 1, wherein
(a) said first nucleic acid portion has a nucleobase sequence selected from SEQ ID NOs: 1 to 30;
(b) said second nucleic acid portion has a nucleobase sequence selected from SEQ ID NOs: 61 to
(c) said third nucleic acid portion has a nucleobase sequence selected from SEQ ID NOs: 31 to 60; and/or
(d) said fourth nucleic acid portion has a nucleobase sequence selected from SEQ ID NOs: 91 to 120.

10. The construct according to claim 1, wherein said first nucleic acid portion of (a) is directly or indirectly linked to said fourth nucleic acid portion of (d) as a primary structure,
said second nucleic acid portion of (b) is directly or indirectly linked to said third nucleic acid portion of (c) as a primary structure,
said first nucleic acid portion has the nucleobase sequence of SEQ ID NO: 3, and/or said second nucleic acid portion has a nucleobase sequence selected from SEQ ID NOs: 61, 66, 67, 73, 68 and 76, and/or said third nucleic acid portion has the nucleobase sequence of SEQ ID NO: 33, and/or said fourth nucleic acid portion has a nucleobase sequence selected from SEQ ID NOS: 91, 96, 97, 103, 98 and 106.

11. (canceled)

12. (canceled)

13. (canceled)

14. The construct according to claim 1, that further comprises_1 to 8, advantageously 2, additional nucleic acid portions that are respectively at least partially complementary to an additional 1 to 8 portions of RNA transcribed from one or more target genes, which target genes may be the same or different to each other, and/or the same or different to the target genes defined in (a) and/or (b), and wherein each of the 1 to 8 additional nucleic acid portions respectively form additional duplex regions with respective passenger nucleic acid portions that are respectively at least partially complementary therewith, said second nucleic acid portion of (b), and said 1 to 8 additional nucleic acid portions, are directly or indirectly linked to selected passenger nucleic acid portions as respective primary structures.

15. (canceled)

16. The construct according to claim 14, wherein said direct or indirect linking represents either (i) an internucleotide bond, said linking is direct, thereby giving rise to (a) contiguous strand(s), there exists some complementarity between the first nucleic acid portion of (a) and the second nucleic acid portion of (b), or the third nucleic acid portion of (c) and the fourth nucleic acid portion of (d), said complementarity
(i) is/are 1, 2, 3, 4, 5, 6, 7, 8, 9, or 10, advantageously 2, 3, 4 or 5 base pairs; and/or
(ii) is between the first nucleic acid portion of (a) and the second nucleic acid portion of (b), said internucleotide bond involves at least one of said one or more unmodified nucleotides, wherein advantageously cleavage occurs at the 3′ position of (at least one of) said unmodified nucleotide(s),
or (ii) a nucleic acid linker portion of 1, 2, 3, 4, 5, 6, 7, 8, 9, or 10 nucleotides, said nucleic acid linker advantageously being single stranded.

17. (canceled)

18. (canceled)

19. (canceled)

20. (canceled)

21. The construct according to claim 1, wherein said first nucleic acid portion of (a), and/or said second nucleic acid portion of (b), and/or said third nucleic acid portion of (c), and/or said fourth nucleic acid portion of (d), are respectively 7 to 25 nucleotides in length,
first nucleic acid portion of (a) and/or said second nucleic acid portion of (b) have a length of 18 to 21, more advantageously 18 to 20, and yet more advantageously 19 nucleotides,
and/or wherein said third nucleic acid portion of (c), and/or said fourth nucleic acid portion of (d) have a length of 11 to 20, more advantageously 13 to 16, and yet more advantageously 14 or 15, most advantageously 15 nucleotides,
said unmodified nucleotide(s) is/are at any of position 18 to 25, more advantageously at any of positions 18 to 21 and/or the 3′ terminal position of said first nucleic acid portion of (a) and/or of said third nucleic acid portion of (c), advantageously unmodified nucleotide is at position 19.

22. (canceled)

23. (canceled)

24. (canceled)

25. (canceled)

26. The construct according to claim 16, wherein said nucleic acid linker portion is 1 to 8 nucleotides in length, advantageously 2 to 7 or 3 to 6 nucleotides in length, more advantageously about 4 or 5 and most advantageously 4 nucleotides in length,
one, more of all of the duplex regions independently have a length of 10 to 19, more advantageously 13 to 19, and yet more advantageously 13, 14 or 15 base pairs, most advantageously 15 base pairs, wherein optionally there is one mismatch within said duplex region,
further comprises one or more ligands, one or more ligands are any cell directing moiety, such as lipids, carbohydrates, aptamers, vitamins and/or peptides that bind cellular membrane or a specific target on cellular surface, one or more ligands are conjugated at the 3′ region,
advantageously the 3′ end, of any of (i) said third nucleic acid portion of (c), and/or (ii) said fourth nucleic acid portion of (d), and/or, to the extent present, said (iii) passenger nucleic acid portions,
said first nucleic acid portion of (a), and/or said second nucleic acid portion of (b), and/or said third nucleic acid portion of (c), and/or said fourth nucleic acid portion of (d), and/or, to the extent present, said 1 to 8 additional nucleic acid portions, and/or said passenger nucleic acid portions, respectively have a 51 to 31 directionality thereby defining 51 and 31 regions thereof.

27. (canceled)

28. (canceled)

29. (canceled)

30. (canceled)

31. (canceled)

32. (canceled)

33. (canceled)

34. The construct according to claim 26, wherein said one or more carbohydrates can be a monosaccharide, disaccharide, tri saccharide, tetrasaccharide, oligosaccharide or polysaccharide,
said one or more carbohydrates comprise one or more hexose moieties,
said one or more hexose moieties are one or more galactose moieties, one or more lactose moieties, one or more N-Acetyl-Galactosamine moieties, and/or one or more mannose moieties, advantageously comprises two or three N-Acetyl-Galactosamine moieties,
and/or one or more ligands are attached in a linear configuration, or in a branched configuration, one or more ligands are attached as a biantennary or triantennary configuration, or as a configuration based on single ligands at different positions, advantageously said ligand has the following structure:

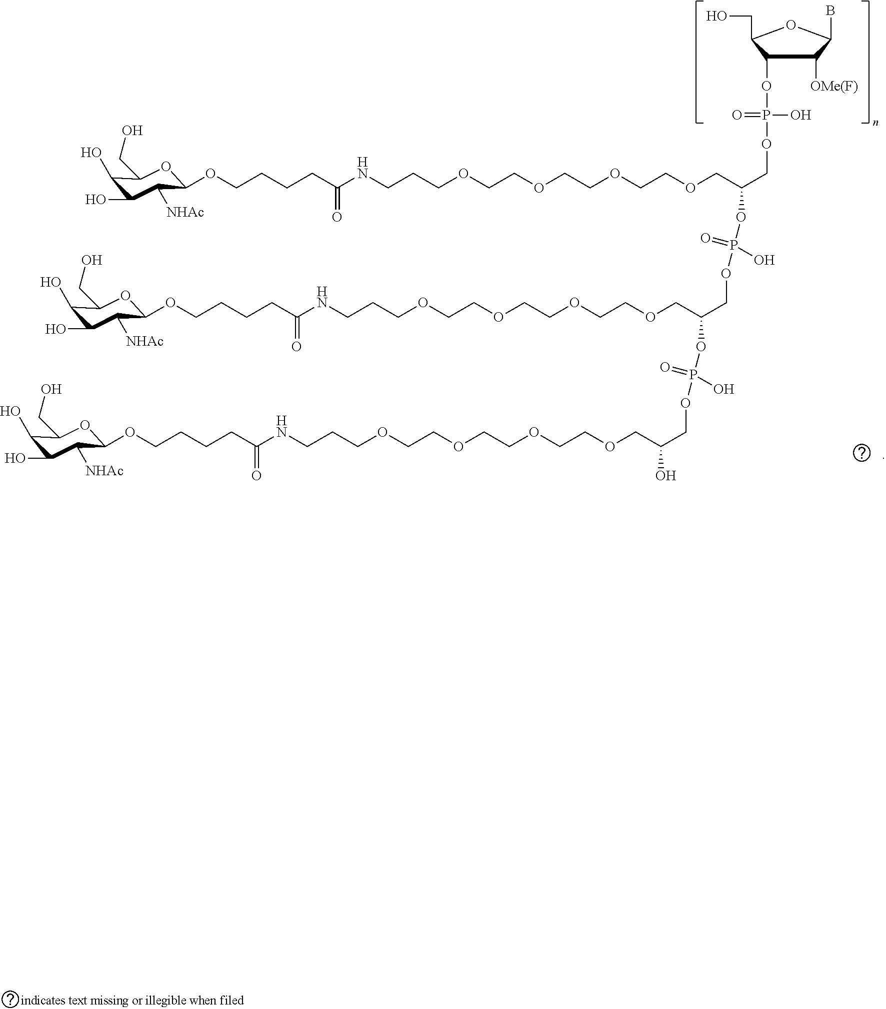

35. (canceled)

36. (canceled)

37. (canceled)

38. (canceled)

39. (canceled)

40. (canceled)

41. (canceled)

42. (canceled)

43. The construct according to claim 34, which comprises one or more phosphorothioate or phosphorodithioate internucleotide linkages at one or more of the 51 and/or 31 regions of said first nucleic acid portion of (a), and/or said second nucleic acid portion of (b), and/or said third nucleic acid portion of (c), and/or said fourth nucleic acid portion of (d), and/or said 1 to 8 additional nucleic acid portions, and/or said passenger nucleic acid portions, and/or comprises phosphorothioate or phosphorodithioate internucleotide linkages between at least two adjacent nucleotides of the nucleic acid linker portion.

44. (canceled)

45. (canceled)

46. (canceled)

47. The construct according to claim 16, wherein at least one nucleotide of at least one of the following is modified: the first nucleic acid portion of (a); and/or the second nucleic acid portion of (b); and/or the third nucleic acid portion of (c); and/or the fourth nucleic acid portion of (d); and/or to the extent present, the 1 to 8 additional nucleic acid portions; and/or to the extent present, the passenger nucleic acid portions; and/or to the extent present, the nucleic acid linker portion.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. The construct according to claim 47, wherein the modification and/or modifications are each and individually sugar, phosphate, or base modifications, advantageously the modification is selected from nucleotides with 2′ modified sugars; conformationally restricted nucleotides (CRN) sugar such as locked nucleic acid (LNA), (S)-constrained ethyl bicyclic nucleic acid, and constrained ethyl (cEt), tricyclo-DNA;
morpholino, unlocked nucleic acid (UNA), glycol nucleic acid (GNA), D-hexitol nucleic acid (HNA), and cyclohexene nucleic acid (CeNA),
said 2′ modified sugar is selected from 2′-O-alkyl modified sugar, 2′-O-methyl modified sugar, 2′-O-methoxy ethyl modified sugar, 2′-O-allyl modified sugar, 2′-C-allyl modified sugar, 2′-deoxy modified sugar such as 2′-deoxy ribose, 2′-F modified sugar, 2′-arabino-fluoro modified sugar, 2′-O-benzyl modified sugar, 2′-amino modified sugar, and 2′-O-methyl-4-pyridine modified sugar,
at least one modification is a 2′-O-methyl modification in a ribose moiety,
at least one modification is a 2′-F modification in a ribose moiety.

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. The construct according to claim 1, wherein said construct comprises a first strand, and a second strand, wherein the nucleobase sequences of said first and said second strands are SEQ ID NO: 130 and 131, SEQ ID NO: 132 and 133, SEQ ID NO: 134 and 135, SEQ ID NO: 136 and 137, SEQ ID NO: 138 and 139, SEQ ID NO: 140 and 141, SEQ ID NO: 142 and 143, SEQ ID NO: 144 and 145, or SEQ ID NO: 146 and 147, respectively.

74. The construct according to claim 73, wherein the construct is selected from the group consisting of SEQ ID Nos. 148-165.

75. (canceled)

76. (canceled)

77. (canceled)

78. (canceled)

79. The construct according to claim 1, which is blunt ended, the target RNA is an mRNA or another RNA molecule.

80. (canceled)

81. (canceled)

82. (canceled)

83. (canceled)

84. A pharmaceutical composition comprising a nucleic acid construct according to claim 1.

85. The pharmaceutical composition of claim 84, further comprising a pharmaceutically acceptable excipient, diluent, antioxidant, and/or preservative, and/or said pharmaceutical composition furthermore comprises one or more further pharmaceutically active agents,
said further pharmaceutically active agents are selected from Vascepa; Vupanorsen; statins such as Rosuvastatin and Simvastatin; fibrates such fenofibrate; LDL-cholesterol lowering compounds such as statins and ezetimib, and/or
agents which decrease hypertension, wherein said further pharmaceutically active agent(s) is/are optionally selected from the group consisting of a diuretic, an angiotensin converting enzyme (ACE) inhibitor, an angiotensin II receptor antagonist, a beta-blocker, a vasodilator, a calcium channel blocker, an aldosterone antagonist, an alpha2-agonist, a renin inhibitor, an alpha-blocker, a peripheral acting adrenergic agent, a selective DI receptor partial agonist, a nonselective alpha-adrenergic antagonist, a synthetic, a steroidal antimineralocorticoid agent; a combination of any of the foregoing; and a hypertension therapeutic agent formulated as a combination of agents, more optionally an angiotensin II receptor antagonist selected from the group consisting of losartan, valsartan, olmesartan, eprosartan, and azilsartan.

86. (canceled)

87. (canceled)

88. (canceled)

89. (canceled)

90. (canceled)

91. (canceled)

92. (canceled)

93. (canceled)

94. A method of treating a disease or disorder comprising administration of a nucleic acid construct according to claim 1, to an individual in need of treatment.

95. The method according to claim 94, wherein the nucleic acid construct is administered subcutaneously or intravenously to the individual,
said disease or disorder is a disease or disorder associated with APOC3 and/or AGT or a disease or disorder requiring reduction of APOC3 and/or AGT expression,
said disease or disorder is selected from the group consisting of an APOC3-associated disease or disorder, or a disease or disorder requiring reduction of APOC3 expression levels, said disease or disorder advantageously being selected from dyslipidemia including mixed dyslipidemia;
hyperchylomicronemia including familial hyperchylomicronemia; hypertriglyceridemia, advantageously severe hypertriglyceridemia and/or hypertriglyceridemia with blood triglyceride levels above 500 mg/dl; inflammation including low-grade inflammation; atherosclerosis;
atherosclerotic cardiovascular diseases (ASCVD) including major adverse cardiovascular events (MACE) such as myocardial infarction, stroke and peripheral arterial disease; pancreatitis including acute pancreatitis; and/or
high blood pressure, hypertension, borderline hypertension, primary hypertension, secondary hypertension isolated systolic or diastolic hypertension, pregnancy-associated hypertension, diabetic hypertension, resistant hypertension, refractory hypertension, paroxysmal hypertension, renovascular hypertension, Goldblatt hypertension, ocular hypertension, glaucoma, pulmonary hypertension, portal hypertension, systemic venous hypertension, systolic hypertension, labile hypertension; hypertensive heart disease, hypertensive nephropathy, atherosclerosis, arteriosclerosis, vasculopathy, diabetic nephropathy, diabetic retinopathy, chronic heart failure, cardiomyopathy, diabetic cardiac myopathy, nocturnal hypotension, glomerulosclerosis, coarctation of the aorta, aortic aneurism, ventricular fibrosis, heart failure, myocardial infarction, angina, stroke, renal disease, renal failure, systemic sclerosis, intrauterine growth restriction (IUGR), fetal growth restriction, obesity, liver steatosis/fatty liver, non-alcoholic Steatohepatitis (NASH), non-alcoholic fatty liver disease (NAFLD); glucose intolerance, type 2 diabetes, and metabolic syndrome.

96. Use of a nucleic acid construct according to claim 1, for use in research as a gene function analysis tool, or for use in the manufacture of a medicament for a treatment of a disease or disorder.

97. (canceled)
